(19) United States
(12) Patent Application Publication
(10) Pub. No.: US 2019/0351227 A1
(43) Pub. Date: Nov. 21, 2019

(54) Method for Relaxing Spasmed Muscles

(71) Applicants: Gennady Petrovich FESKOV, Saint Petersburg (RU); Anna Yurievna LITUS, Saint Petersburg (RU)

(72) Inventors: Gennady Petrovich FESKOV, Saint Petersburg (RU); Anna Yurievna LITUS, Saint Petersburg (RU)

(21) Appl. No.: 16/476,140

(22) PCT Filed: Jan. 23, 2018
(86) PCT No.: PCT/RU2018/000021

§ 371 Date: Jul. 5, 2019

(30) Foreign Application Priority Data
Jan. 23, 2017 (RU) 2017102164

(51) Int. Cl. A61N 1/36

(52) U.S. Cl. CPC A61N 1/3603; A61N 1/28; A61N 1/36003

(57) ABSTRACT

The invention relates to medicine, and more particularly to physiotherapy, neurology, traumatology, orthopaedics, rehabilitation and sports medicine, and can be used for relaxing spasmed muscles when treating patients with different conditions of the musculoskeletal and nervous systems, and also for prophylaxis in the social sphere. For this purpose, relaxed antagonist muscles are subjected to a stimulating electric impulse effect at an impulse frequency of from 15 to 35 Hz, with an impulse signal duration of from 5 to 60 μs and a working signal level of from 5 to 75% of the strength of a signal at which the patient feels a vibration. Spasmed agonist muscles are subjected to a relaxing thermal effect at a temperature of from 42 to 60° C. In addition, the segments of the spinal cord responsible for innervating the agonist-antagonist muscle pairs undergoing treatment are subjected to a thermal effect at a temperature of from 42 to 60° C. The agonist-antagonist muscle pairs and the segments of the spinal cord responsible for innervating the agonist-antagonist muscle pairs can be acted on simultaneously and alternately. The stimulating electric impulse effect and the relaxing effect can be carried out on individual regions of the body or on all regions simultaneously. The method provides effective physiological relaxation of spasmed muscles by means of a synergistic effect resulting from a combination of effects which engage the central nervous system that regulates and alters the physiological state of the tissues of agonist-antagonist muscle pairs.

### TECHNICAL FIELD

The invention relates to medicine, and in particular to physiotherapy, neurology, traumatology, orthopaedics, rehabilitation and sports medicine, and is intended for relaxing spasmed muscles when treating patients with different conditions of the musculoskeletal and nervous systems, and also for prophylaxis in the social sphere.

The invention can be used to relieve pathological muscle spasms, restore the physiological structure of the musculoskeletal system, relieve the pain syndrome during myospasms, prevent aggravation of destructive processes as a result of frequent and prolonged myospasms, restore the patient's normal motor activity and adapt him/her to the social and living conditions of life.

The invention can be used to treat the following diseases:
in neurology:
spastic muscle and joint deformities in infantile cerebral paralysis,
conditions after injuries of the musculoskeletal system,
pain syndromes associated with spasms of skeletal muscles in various parts of the human body, including central genesis,
abnormalities of the statics and dynamics of the spine cord associated with the myospastic syndrome,
vertebral myalgia (myofascial syndromes),
congenital anomalies of the musculoskeletal system accompanied by a chronic pain syndrome,
pain associated with a “weak posture”;
in sports medicine:
pathological overstrain of skeletal muscles as a result of intense sports loads,
preventive tonus adaptation of the musculo-ligamentous apparatus to subsequent physical exertion,
comprehensive rehabilitation after sports injuries;
in the social sphere:
preventive tonus regulation after work-associated physical exertion (in functional load release rooms),
to improve the quality of life, in particular, for normalization of sleep in the conditions of boarding houses, rest homes, elderly care institutions.

### BACKGROUND ART

There is a known method of relaxation of spasmed muscles, which includes stimulating electropulse effect on a relaxed antagonist muscle, as well as a vibratory stimulating effect on the ligaments, the joint capsule or the tendon, through which the spasmed agonist muscle is attached to the spine cord (see WO 2011/067327, publ. Sep. 6, 2011).

The disadvantage of this method is the fact that the vibration refer is a factor characterized by high biological activity. Vibration produces a stimulating effect, thus, when stimulating the spastic agonist muscle, the process of its further compression, and, as a result, mutual inhibition (relaxation) of the already-relaxed antagonist muscle occurs, therefore spasmodic muscle relaxation does not occur.

In addition, local vibration causes vascular spasms, disrupting the blood supply to the extremities, causes a decrease in skin sensitivity, deforms and reduces mobility of the joints. Local vibration also disrupts the activity of the central nervous system. That is explained by the fact that vibration is capable of propagation, affecting the nervous and musculoskeletal systems. Prolonged vibration impacts the cardiovascular system and especially the microcirculatory bloodstream (small vessels in which the immediate release of oxygen from the blood and utilization of carbon dioxide from the tissues take place).

Moreover, vibration at a frequency of 6-9 Hz, close to the natural frequency of the internal organs, can lead to resonance. The impact of general vibration in the resonant zone is very dangerous, since it can cause mechanical damage to human internal organs, lead to mechanical tissue breaks and internal hemorrhages.

These circumstances determine the lack of effectiveness of relaxation of spasmed muscles, as well as the non-physiological character of the treatment.

There is a method of relaxation of spasmed muscles, which includes stimulating (vasoactive) electropulse effect of a sequence of electrical impulses onto relaxed antagonist muscles, as well as a relaxing electric impulse effect onto spasmed agonist muscles produced by a sequence of electrical impulses suitable for inhibition of the H-reflex phase. Pulse sequences are fed independently from each other through channels to areas of the body (antagonist and agonist muscles). The parameters of the pulses are regulated based on the degree of hypertension or hypotonia of the patient's muscles (see RU 2438732 C2, publ. Oct. 1, 2012).

This method is adopted as a prototype of the claimed invention.

The prototype has the following disadvantages:
the difficulty of selecting the parameters of electrical impulses suitable for initiating the depression of the H-reflex phase in spasmed muscles;
the need for long-term electropulse effects in order to adapt the spasmed agonist muscles for relaxation, since initially, the effect of electrical impulses on muscles is stimulating, and the process of further compression of spasmed muscles takes place, and only after adaptation to impulses with certain parameters, their relaxation can occur;
the non-physiological nature of the treatment due to the need for prolonged exposure to the electric impulse effect in order to adapt spasmed agonist muscles for relaxation. A prolonged exposure to the electric impulse effect has a harmful impact on the body.

### SUMMARY OF THE INVENTION

It is an object of the present invention to create an effective, physiological (attenuated) method of relaxation of spasmed muscles, which does not adversely affect the human body.

The task is solved by virtue of the fact that in the method of relaxation of spasmed muscles including a stimulating electric impulse effect on relaxed antagonist muscles and a relaxation effect on spasmed agonist muscles, according to the invention, the spinal segments responsible for innervation of the agonist/antagonist muscle pairs to be treated are additionally subjected to thermal effect of 420° C. to 600° C.

The applicant has not identified any engineering solutions that are identical to the claimed one, which allows to make a conclusion about the compliance of the invention with the patentability criterion of “Novelty” (“N”).

The relaxation effect on the spasmed agonist muscles is carried out mainly by exposure to thermal effect of 420° C. to 600° C., while relaxed antagonist muscles are subjected to a stimulating electric impulse effect at an impulse frequency of 15 to 35 Hz, with impulse signal duration of 5 to 60 μs and working signal level of 5 to 75% of the strength of the signal at which the patient feels vibration. The agonist/antagonist muscle pairs and the segments of the spinal cord responsible for innervating the agonist/antagonist muscle pairs can be acted on simultaneously or alternately. The agonist/antagonist muscle pairs can be acted on alternately in the setting of a constant effect on the segments of the spinal cord responsible for innervating the agonist/antagonist muscle pairs. The stimulating electric impulse effect and the relaxing effect can be carried out on individual regions of the body or on all of the abovementioned body regions simultaneously.

The combination of the stimulating electric impulse effect on relaxed antagonist muscles, a relaxation effect on spasmed agonist muscles and effects on the segments of the spinal cord responsible for innervating the agonist/antagonist muscle pairs to be treated allows to bring into action the central nervous system, which regulates and alters the physiological state of the tissue of agonist/antagonist muscle pairs, adapting them to ongoing activities. Thereby, a synergistic effect is created from a combination of the three effects, which ensures effective relaxation of spasmed muscles.

The relaxation effect on the spasmed agonist muscles and the exposure of the segments of the spinal cord to heat (thermal effect) does not adversely affect the human body, i.e. is a physiological method of action. In addition, heat penetrates deep into tissues and muscle structures, improves their nutrition, blood supply and innervation, has a wide coverage of the surface to be treated and a long post-effect, which also ensures effective relaxation of spasmed muscles.

The possibility of affecting agonist/antagonist muscle couples simultaneously, or alternately with the impact on the spinal segments responsible for the innervation of the agonist/antagonist muscle pairs, or alternately in the setting of a constant effect on the segments of the spinal cord responsible for innervating the agonist/antagonist muscle pairs allows for selection of the most effective way of relaxation individually for a group of muscles and for a particular person.

The possibility of exposure of local areas of the body or all segments of the body at the same time also allows for selection of the most effective way of relaxation, depending on the number of spasmed muscles to be treated.

The claimed method is implemented using a device for relaxation of spasmed muscles that contains a set of electrodes grouped in three.

The first electrode is made with the possibility of imposing on a relaxed antagonist muscle to supply a stimulating electric impulse effect. The chosen parameters of the pulse current signal are close to physiological. It was experimentally shown that muscles begin to contract at a pulse frequency of 15 Hz to 35 Hz. The chosen duration of the pulse current signal is close to the duration of the nerve signal impulses—5 μs to 60 μs. The current signal level is chosen so that it is in the range of the signal strength of 5% to 75%, at which the patient feels the vibration.

The second and third electrodes are made with the possibility of imposing on the spasmed agonist muscle and on the segment of the spinal cord responsible for the innervation of the agonist/antagonist muscle pair to be treated, to provide thermal effect. At that the temperature of exposure is chosen in accordance with the human neurophysiological parameters, so as not to cause discomfort to the patient. It was experimentally shown that the muscles relax at a temperature of 420° C. to 600° C.

It should be borne in mind that thermal effect on the spasmed agonist muscle and on the spinal segment can be carried out by any known method, including the application of a hot compress.

The claimed method is implemented as follows.

The relaxed antagonist muscles of the agonist/antagonist muscle pairs to be treated are stimulated by the stimulating electric impulse effect by the pulse frequency of 15 Hz to 35 Hz and duration of 5 μs to 60 μs. The current signal level is chosen individually depending on the sensitivity of the patient so that it is in the range of the signal strength of 5% to 75%, at which the patient feels the vibration.

Spasmed agonist muscles are subjected to the relaxation effect, mainly with heat of 420° C. to 600° C., depending on the sensitivity of the patient. Thermal effects from any heat carrier can be applied, for example, an electric or light heater, light heating, application of wax baths, heating pads, hot compresses can be used.

The segments of the spinal cord responsible for innervating the agonist-antagonist muscle pairs undergoing treatment are affected mainly by heat by a temperature of 420° C. to 600° C., depending on the sensitivity of the patient.

The agonist/antagonist muscle pairs and the segments of the spinal cord responsible for innervating the agonist/antagonist muscle pairs can be acted on simultaneously or alternately. The agonist/antagonist muscle pairs can be acted on alternately in the setting of a constant effect on the segments of the spinal cord responsible for innervating the agonist/antagonist muscle pairs. The stimulating electric impulse effect and the relaxing effect can be carried out on individual regions of the body or on all of the body regions simultaneously.

Stimulation of antagonist muscles causes their contraction, which causes relaxation of the corresponding spasmed agonist muscles. The relaxation effect on spasmed agonists muscles helps to relax them.

Inclusion of exposure of the spinal segments responsible for the innervation of agonist/antagonist muscle pairs into the process of interaction of agonist/antagonist muscle pairs provides for excitation of the corresponding segments of the central nervous system, whose role is to regulate metabolism, excitability and automatism of peripheral organs.

At the same time, the nervous system regulates and changes the physiological state of the tissues of agonist/antagonist muscle pairs, adapting them to the current activity, which ensures effective relaxation of spasmed muscles.

The muscles to be treated are selected by the doctor for each patient, depending on the indications.

Each spasmed agonist muscle to be treated must be paired with an appropriate antagonist muscle, which must be associated with a segment of the spine cord responsible for innervation of the chosen agonist/antagonist muscle pair.

The applicant has not identified any sources of information that would contain information about the effect of the distinctive features of the invention on the technical result achieved. The abovementioned circumstances allow for the conclusion about the compliance of the claimed technical solution to the patentability criterion of “Inventive step” (“IS”).

### BRIEF DESCRIPTION OF THE DRAWINGS

The invention is further explained in the detailed description of examples of its implementation with reference to the drawings, on which:

FIG. 1 shows the areas of exposure of the agonist/antagonist muscle pairs, where the numbers correspond to the muscles, in accordance with Table 1.

FIG. 2 presents the scheme of interconnections between spinal segments and the organs and body parts, for the innervation of which they are responsible, in accordance with Table 2.

FIG. 3 shows the areas of exposure of the agonist/antagonist muscle pairs and spinal segments as per Example 1.

FIG. 4 shows the areas of exposure of the agonist/antagonist muscle pairs and spinal segments as per Example 2.

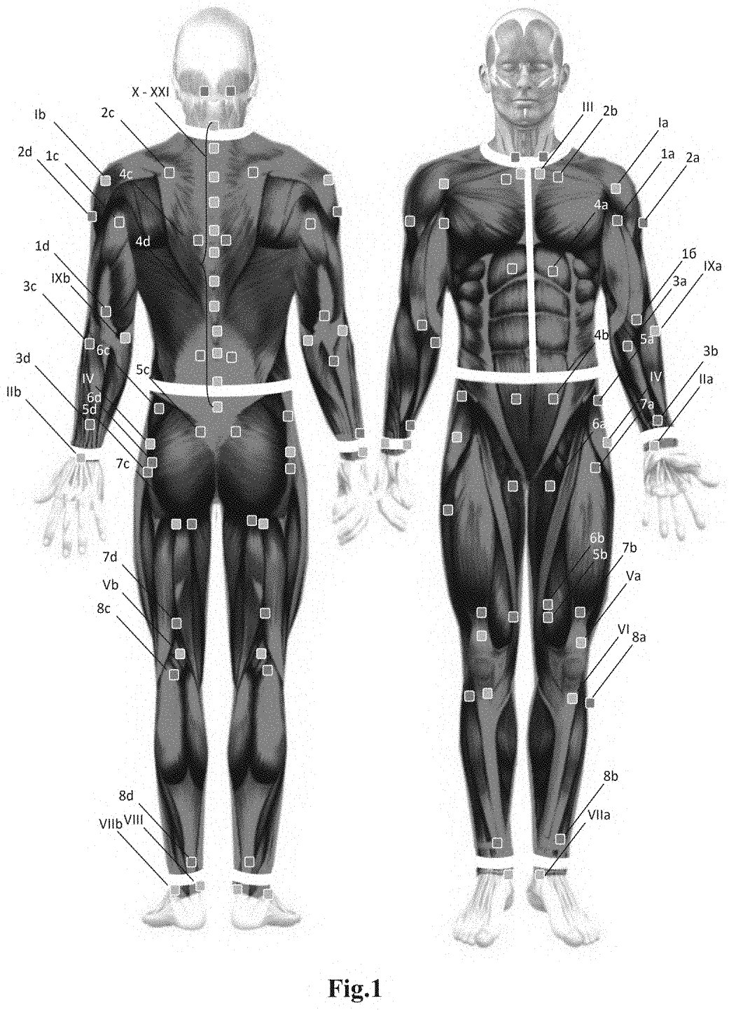

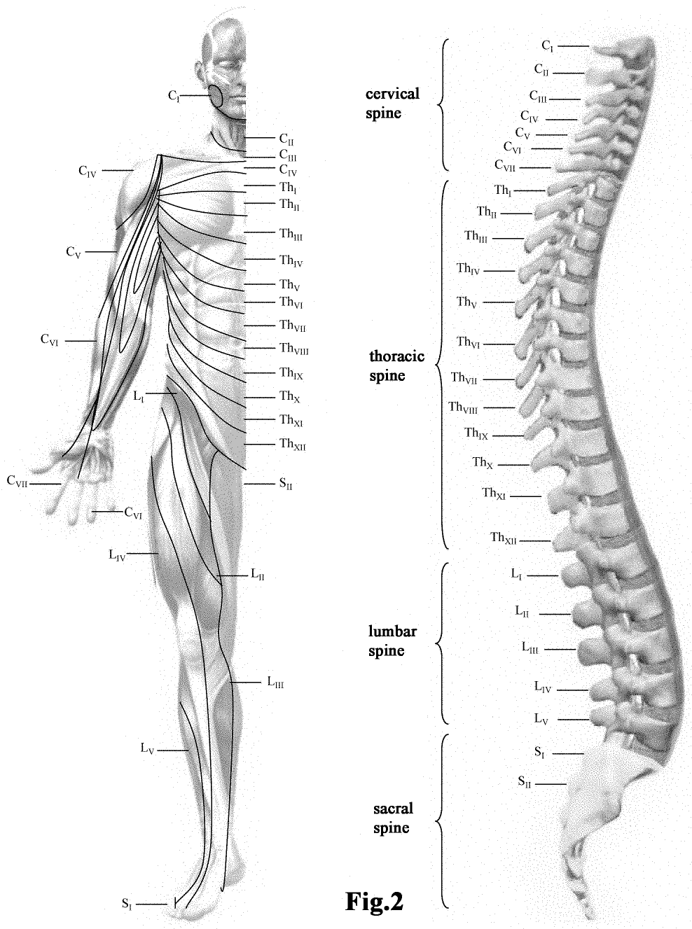

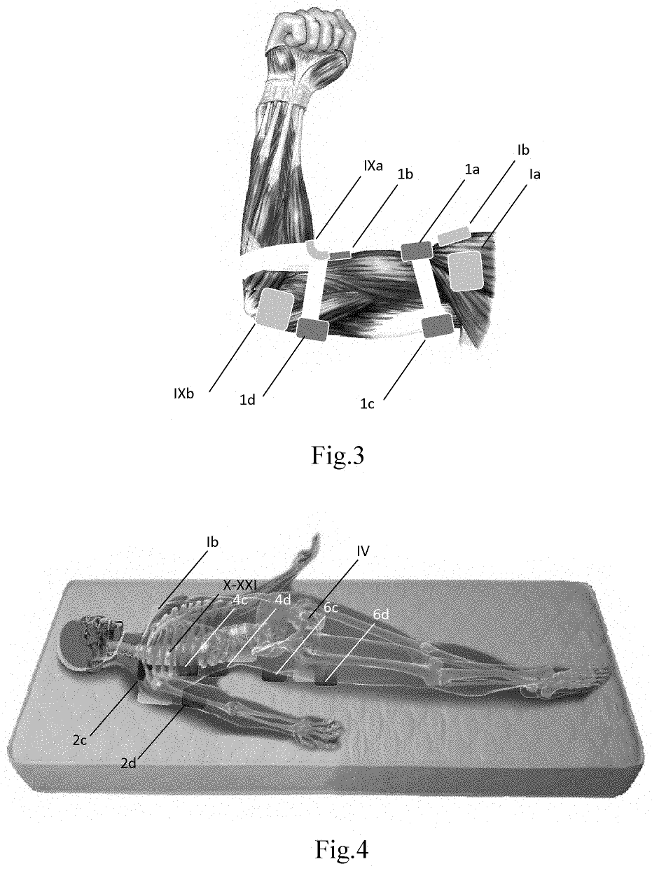

### PREFERRED EMBODIMENT

Table 1 shows the names of the areas of exposure of the agonist/antagonist muscle pairs.

Table 2 presents the schemes of interconnections between spinal segments and the organs and body parts, for the innervation of which they are responsible.

Table 3 shows the schemes of interconnections between agonist muscles, which are subjected to relaxing, in particular thermal, effects, antagonist muscles, which are stimulated by electric impulse effects, and the spinal segments responsible for the innervation of the agonist/antagonist muscle pairs which are thermally affected.

Implementation of the invention is illustrated by examples.

In the examples, the electric impulse effect is carried out by means of the first electrode, and the relaxation effect, mainly thermal, is carried out by means of the second electrode.

The above location and the alphanumeric designation of muscle stimulation areas indicate consistent groups of agonist muscles and their antagonists. Stimulation of antagonist muscles may be used to reduce spasmedity of agonist muscles, and in the case of atony, muscle weakness or paralysis discrete stimulation of agonist muscles and antagonist muscles may be used. Performed in the abovementioned local areas, exposure to the electrode effects has a stimulating effect not only on a single muscle or its separate myogaster (venter) or head (caput), as is the case with muscles having several myogasters (m.biceps brachii, m.triceps brachii, in.quadriceps femoris, in.quadriceps femoris . . . ) or having parts that make up a muscle (m.pectoralis, m.gluteus . . . ), but also on the whole muscle and its parts, having a general stimulating effect. At the same time, a group of muscles that perform a function of the same type, for example, extension (agonists), are also stimulated either by direct influence, when located in the zone of the electrode influence, or indirectly, reflexively, thus exerting a relaxing effect through inhibiting intercalary neurons of their spinal cord segments onto the matching antagonist muscle group, which leads to a consistent, adequate balance. Thus, there is no need to impose electrodes on all muscles available.

To reduce tonicity of agonist muscles (or group thereof), not only the combined antagonist muscles, but also the antagonist muscles also participating in the performance of the same function can be simultaneously stimulated.

### Example 1

The patient is a 50 year old man. The spasmed muscle is the biceps muscle of one of the arms.

At the same time, spinal segments (CV-CVII) were acted upon by application of a warming compress at a temperature of 450° C. to 600° C. and on agonist/antagonist muscle pairs.

To stimulate muscles and heat the corresponding joints and ligaments, local influence by means of electrodes was used. In this example, a cuff (for example, with touch fasteners) is used, which was put on the shoulder with the location of the electrodes as described:

Agonist Muscle:

1a—the place of application of the electrode is the proximal part of the myogaster of the biceps muscle of one of the arms (venter) m.biceps brachii) at the place of its transition into the tendon (tendo m.biceps brachii) innervation (CV-CVII),

1b—the place of application of the electrode is the distal part of the myogaster of the biceps muscle of one of the arms (venter) m.biceps brachii) innervation (CV-CVII),

Ia—the place of application of the heating element is localized by the point of attachment of the short head of the biceps muscle of the arm (caput breve m.biceps brachii) to the coracoid process of the scapula (processus coracoideus scapula), as well as the front surface of the shoulder joint (articulation humeri) and the adjacent glenohumeral, coracoid-acromial and coraco-humeral ligaments (ligg.glenohurale superius, mediale, inferius; lig.coracoacromeale; lig.coracohumorale); the tendon of the biceps muscle of the arm (tendo m.brahii (caput breve)); joint capsule (capsula articularis),

Ib—the place of application of the heating element is localized by the point of attachment of the long head of the biceps muscle of the arm (caput longum m.triceps brachii) to the articular tubercle of the scapula (tuberculum infraglenoidale angulus lateralis scapule), as well as the posterior surface of the shoulder joint (articulation humeri) and the transverse and beak-humeral ligaments adjacent to it (lig.transversum scapule; lig.coracohumorale); tendon of the long head of the triceps muscle of the arm (caput longum m.triceps brachii); joint capsule (capsula articularis), subacromial bursa (bursa subacromialis);

Antagonist Muscle:

1c—the place of application of the electrode is the place of passage of the long head of the triceps muscle of the arm (caput longum m.triceps brachii) innervation (CV-CVII),

IXa—the place of application of the heating element is localized by the place of attachment of the myogaster of the biceps of the arm (venter m.biceps brachii) to the tuberosity of the radial bone (tuberositas radii), as well as the humeroradial and humeroulnar joints (articulatio humeroradialis, articulatio humero-radialis) and the adjacent collateral radial ligament (lig.collaterale radiale), the annular radial ligament (lig.anulare radii), the square ligament (lig.quadratum); the tendon of the biceps muscle of the arm (tendo m.brachii (caput breve)); joint capsule (capsula articularis),

1d—the place of application of the electrode is the tendon of the triceps muscle of the arm (tendo m.triceps brachii) innervation (CV-CVII),

IXb—the place of application of the heating element is localized by the place of attachment of the tendon of the triceps muscle of the arm (tendo m.triceps brachii) to the ulnar process of the ulna (olecranon ulnaris), as well as the surface of the pomelolecteal joint (articulatio humeroulnaris) and the adjacent ulnar collateral ligament (lig. collaterale ulnare), the radial collateral ligament (lig.collaterale radiale); the intratendinous bursa (bursa intratendinea olecrani); the subcutaneous bursa of the olecranon (bursa subcutanea olecrani); the tendon of the triceps muscle of the arm (tendo m.triceps brachii); joint capsule (capsula articularis).

As a result of the five sessions, the spasmed muscle was relaxed.

### Example 2

The patient is a 60 year old man. The patient is in supine position, unable to get up. The dorsal (extensor) muscles of the trunk are spasmed.

Segments (CV-CVII) of the cervical spine, segments (ThI-ThXII) of the thoracic spine, segments (LI-LV) of the lumbar spine and the SI segment of the sacral spine were constantly affected by applying a warming compress at a temperature of 45° C. to 60° C. The agonist/antagonist muscle pairs were acted on alternately in the setting of a constant effect on the segments of the spinal cord.

This example illustrates an option of the zonal application of electrodes, for example, embedded in the surface of the mattress, with the ability to move them in planes, which can be fixed, for instance, using touch fasteners. In this example, the following electrodes were used:

agonist muscles: deltoid muscle (venter m.deltoideus) and greater pectoral muscle; (mm pectoralis major);

innervation (CV-CVII);

antagonist muscles:

2c—the place of application of the electrode is the proximal part of the posterior myogaster of the deltoid muscle (venter m.deltoideus posterior) and the proximal part of the circular muscle (mm teres major et minor) innervation (CV-CVII)

2d—the place of application of the electrode is the distal part of the myogaster of the deltoid muscle (venter m.deltoideus) and the distal part of the myogaster of the circular muscle (mm teres major et minor) innervation (CV-CVII)

Ib—the place of application of the heating element is localized by the point of attachment of the long head of the triceps muscle of the arm (caput longum m.triceps brachii) to the articular tubercle of the scapula (tuberculum infraglenoidale angulus lateralis scapule), as well as the posterior surface of the shoulder joint (articulation humeri) and the transverse and beak-humeral ligaments adjacent to it (lig.transversum scapule; lig.coracohumorale); the tendon of the long head of the triceps muscle of the arm (caput longum m.triceps brachii); joint capsule (capsula articularis), the subacromial bursa (bursa subacromialis), the crest of the lesser tubercle of humeri of the humerus (crista tuberculi minor).

Agonist muscle: the rectus abdominis muscle (m.rectus abdominis),

innervation (ThV-ThXII, LI),

antagonist muscles:

4c—the place of imposition of the electrode is the rib part of the longissimus dorsi (in longissimus), the iliocostalis muscle (m.iliocostalis), innervation (ThI-LI),

4d—the place of imposition of the electrode is the iliac parts of the longissimus dorsi (in longissimus) and the iliocostalis muscle (m.iliocostalis). innervation (ThI-LI).

agonist muscles: the gluteus maximus muscle (m.gluteus medius) and the wide fascia strainer (m.tensor fasciae latae),

innervation (LII-LV), X-XXI—the place of application of the heating element is localized by the place of the cervical and lumbar spine (CV-LI) and the intervertebral symphysis (symphysis intervertebtralis), articulation joints (articulationes zygapophysiales), and the longitudinal yellow ligaments (ligg. longitudinales et flava) adjacent to them,

antagonist muscles:

6c—the place of application of the electrode is the place of the proximal attachment of the gluteus medius muscle (m.gluteus medius) and the tensor muscle of fascia lata (m.tensor fasciae latae). innervation (LIV-SI)

IV—the place of application of the heating element is localized by the attachment point of the proximal part of the quadriceps muscle of thigh (m.quadriceps femoris), the proximal part of the tailor's muscle of thigh (m.sartorius), the gluteus maximus muscle (m.gluteus maximus); the gluteus medius muscle (m.gluteus medius); the gluteus minimus muscle (m.gluteus minimus); the square muscle of thigh (m.qudratus femoris); the tensor muscle of fascia lata (m.tensor fasciae latae); as well as the surface of the hip joint (articulatio coxae) and the transverse bundle of the acetabulum adjacent to it (lig.transversum acetabuli); the iliofemoral ligament (lig.iliofemorale); the pubofemoral ligament (lig.pubofemorale); the sciatic-femoral ligament (lig.ischiofemorale); the circular zone (zona orbicularis); the ligament of the femoral head (lig.capitis femoris); the tendon of the biceps of the arm (tendo m.brahii (caput breve)); the bursa of the gluteus maximus muscle (bursa trochanterica in. glutei maximi); the intermuscular bursa of gluteus muscles (bursae intermusculares mm gluteorum); the ischial bursa of gluteus maximus (bursa ischiadica m.glutei maximi); the trochanteric bursa of the gluteus maximus muscle (burse trochantericae m.glutei medii); the trochanteric bursa of the gluteus minimus muscle (bursa trochanterica m.glutei minimi); joint capsule (capsula articularis).

6d—the place of application of the electrode is the place of the distal attachment of the gluteus medius muscle (m.gluteus medius) and the transition of the myogaster of the tensor muscle of fascia lata (m.tensor fasciae latae) into the tendon. innervation (LIV-SI)

If necessary, the use of, for example, a mattress and cuffs on a limb can be combined, or the electrodes can be applied separately, for example, on the front part of the body.

After the sessions, the patient gradually began to regain the ability to sit down. Currently, the patient is able to get out of bed by himself.

### Example 3

The patient is a 7 year old boy diagnosed with cerebral palsy in hyperkinetic form.

An asymmetrical tonic neck reflex (ATNR) is expressed: the head is turned to the left, tonicity of the extensor muscles of the extremities on the left is enhanced, and on the right tonicity of the evertor muscles of the extremities is enhanced.

Due to the presence of ATNR and in connection with hyperkinesis of the muscles of the tongue, swallowing processes are difficult, at the time of the beginning of the rehabilitation course self-assisted eating is impossible.

Movement disorders are characterized by involuntary movements and postures, which are represented by hyperkinesis of the torsion dystonia type against the background of increased muscle tone of extrapyramidal type. Dystonic attacks are intensified during voluntary movements, provoked by emotions.

Voluntary movements are awkward, uncoordinated, harsh. The presence of an asymmetrical tonic neck reflex makes it impossible to hold the head in the middle position and bring a hand to the face.

The patient cannot independently support the supine posture. The patient cannot independently turn over to the side or to the pronate position. The patient cannot support the sitting position.

Frequent and strong dystonic attacks make it impossible to acquire any self-service skills or organize any leisure cognitive activities.

The segments of the spinal cord and agonist/antagonist muscle pairs were acted on alternately.

For stimulation, the following antagonist muscles were chosen to induce relaxation in spasmed agonist muscles.

Sternocleidomastoid (m.sternocleidomastoideus)—due to a sharp and strong head turn, the specialist decided to create a sedative heat effect on the cervical spine with a heating element by the temperature of 42° C.

X-XI—the place of application of the heating element was dictated by the region of the cervical spine (C_{V}-C_{VII}).

The pectoralis major (mm pectoralis major) antagonist muscle of the agonist muscle of the posterior myogaster of the deltoid muscle (venter m.deltoideus posterior) and the teres muscle (mm teres major et minor), due to spasmedity of which there is a sharp and strong turn of the head and the body to the left with spinal over-bending. It was decided to stimulate the right side much stronger than the left one. A voltage of 30 μs was set on the left and 55 μs on the right, and a sedative thermal effect with a heating element by the temperature of 42° C. was added.

2a—the place of application of the electrode is the place of the distal myogaster of the deltoid muscle (venter m.deltoideus) and the attachment of the pectoralis major muscle to the humerus (mm pectoralis major), innervation (C_{V}-C_{VI}) in the range of approximately 1 to 1.5 cm is more proximal in relation to the visually defined transition of the deltoid muscle to the lateral part of the shoulder.

Ia—as the place of application of the heating element, the point of attachment of the short head of the biceps muscle of the arm (caput breve m.biceps brachii) to the coracoid process of the scapula (processus coracoideus scapula) was chosen, as well as the attachment of the deltoid muscle (venter m.deltoideus) and the pectoralis major muscle (mm pectoralis major) to the crest of the large tubercle (crista tuberculi majoris), and the front surface of the shoulder joint (articulation humeri) and the adjacent glenohumeral, coracoid-acromial and coraco-humeral ligaments (ligg.glenohurale superius, mediale, inferius; lig.coracoacromeale; lig.coracohumorale); the tendon of the biceps muscle of the arm (tendo m.brahii (caput breve)); joint capsule (capsula articularis), the subdeltoid bursa (bursa subdeltoidea).

2b—as the places of application of the second electrode the following were chosen: the proximal part of the myogaster of the pectoralis major muscle (mm pectoralis major) and the anterior part of the myogaster of the deltoid muscle (venter m.deltoideus anterior) innervation (C_{V}-C_{VI}) in the subclavian fossa approximately in the middle.

III—as the place of application of the heating element the place of attachment of the myogaster of the biceps pectoralis major (mm pectoralis major) to the medial edge of the clavicle (corpus claviculare) in the sternoclavicular joint (articulatio sternoclavicularis) was chosen, as well as the sternoclavicular, costoclavicular and interclavicular ligaments (lig.sternoclaviculare; lig.costaclaviculare, lig.interclaviculare); joint capsule (capsula articularis) adjacent to it.

Triceps (m.triceps brachii) is an antagonist muscle of agonist muscle of the biceps muscle of the arm (m.biceps brachii), in which dystonic attacks frequently occur, extensor muscles of the shoulder on the right and left were stimulated equally—55 μs, and also a sedative heat effect with a heating element by the temperature of 42° C. was added.

1c—as the place of application of the electrode, the place of passage of the long head of the triceps muscle of the arm (caput longum m.triceps brachii) was chosen; innervation (C_{V}-C_{VIII}) of the palpation-defined depression on the dorsal side of the humerus between the muscles of the posterior bundles of the deltoid muscle (m.deltoideus), the teres major muscle (m.teres major).

IXa—as the place of application of the heating element, the place of attachment of the myogaster of the biceps of the arm (venter m.biceps brachii) to the tuberosity of the radial bone (tuberositas radii) was chosen, as well as the humeroradial and humeroulnar joints (articulatio humeroradialis, articulatio humero-radialis) and the adjacent collateral radial ligament (lig.collaterale radiale), the annular radial ligament (lig.anulare radii), the square ligament (lig.quadratum); the tendon of the biceps muscle of the arm (tendo m.brachii (caput breve)); joint capsule (capsula articularis).

1d—as the place of application of the electrode, the tendon of the triceps muscle of the arm (tendo m.triceps brachii) innervation (C_{V}-CVII) was chosen, about 2 cm proximal of the olecranon process (olecranon).

IXb—as the place of application of the heating element, the place of attachment of the tendon of the triceps muscle of the arm (tendo m.triceps brachii) to the ulnar process of the ulna (olecranon ulnaris) was chosen, as well as the surface of the pomelolecteal joint (articulatio humeroulnaris) and the adjacent ulnar collateral ligament (lig. collaterale ulnare), the radial collateral ligament (lig.collaterale radiale); the intratendinous bursa (bursa intratendinea olecrani); the subcutaneous bursa of the olecranon (bursa subcutanea olecrani); the tendon of the triceps muscle of the arm (tendo m.triceps brachii); joint capsule (capsula articularis).

The carpi radialis and ulnar wrist extensors (m.extensor carpi ulnaris) (m.extensor carpi radialis) are antagonist muscles of agonist muscles of the ulnar wrist flexor (m.flexor carpi ulnaris), the radial wrist flexor (m.flexor carpi radialis), and the superficial digital flexor (m.digitorum superfacialis), as a result of hypersthenia of which symmetrical closure of the hands into fists and flexion-pronation synergy of the hands occur; the pulse duration is set equal on the right and on the left—55 μs, and also a sedative heat effect with a heating element by the temperature of 42° C. was added.

3c—as the place of attachment of the electrode, the place of attachment of the myogaster of the ulnar extensor muscle of wrist (m.extensor carpi ulnaris) innervation (C_{VI}-C_{VIII}) was chosen, as well as the radial extensor muscle of the wrist (m.extensor carpi radialis) innervation (C_{V}-C_{VII}), and extensor muscles of the fingers (m.extensor digitorum) innervation (C_{VI}-C_{VIII}), approximately 2 cm distal of the ulnar process (olecranon).

IXb—as the place of application of the heating element, the place of attachment of the myogaster of the ulnar extensor muscle of wrist (m.extensor carpi ulnaris), the radial extensor muscle of the wrist (m.extensor carpi radialis), and extensor muscles of the fingers (m.extensor digitorum) to the ulnar process of the ulna (olecranon ulnaris) and the lateral elbow was chosen, as well as the surface of the pomelolecteal joint (articulatio humeroulnaris) and the adjacent ulnar collateral ligament (lig. collaterale ulnare), the radial collateral ligament (lig.collaterale radiale); the intratendinous bursa (bursa intratendinea olecrani); joint capsules (capsula articularis).

3d—the place located just above the wrist at the transition of the myogaster to the tendon part of the elbow wrist extensor (m.extensor carpi ulnaris) innervation (C_{VI}-C_{VIII}), radial wrist extensor (m.extensor carpi radialis) innervation (C_{V}-C_{VII}), and extensor muscles of the fingers (m.extensor digitorum) innervation (C_{VI}-C_{VIII}) was chosen as the place of application of the electrode, approximately 1-1.5 cm proximal of the radiocarpal joint between the ulnar and radial bones.

Ib—as the places of application of the heating element, the following were chosen: the back of the wrist joint (articulatio radiocarpea) and the distal radioulnar joint (articulatio radioulnaric distalis), and the adjoining back radiocarpal ligament (lig.radiocarpeum dorsale), and the collateral radius and the ulna carpal ligament (ligg. collaterale carpi radiale, collaterale carpi ulnare), the tendons of the elbow extensor of the wrist (m.extensor carpi ulnaris), the radial extensor of the wrist (m.extensor carpi radialis), the finger extensor (m.extensor digitorum); joint capsules (capsula articularis); as well as the tendon sheaths of the extensors of the fingers and the little finger (vagina tendinum mm extensor digitorum et extensoris indicis), the radial tendon of the wrist (vagina tendium mm. extensorum carpi radialium), the ulnar tendon of the wrist (vagina tendinis mm extensor carpi ulnaris), the flexor tendon of the long finger of the hand (vagina tendinis m.extensoris policis longi).

The rectus abdominis muscle (m.rectus abdominis) is the antagonist muscle of agonist muscles of the longissimus dorsi muscle (m.longissimus) and the iliocostalis muscle (m.iliocostalis), in which frequent dystonic attacks and over-bending of the lumbar spine occur—the set stimulation value was the maximum possible for the age and the muscle—50 μs, and a sedative thermal effect with a heating element by the temperature of 42° C. was added.

4a—the upper part of the rectus abdominis muscle (m.rectus abdominis). innervation (Th_{V}-Th_{XII}, L_{I}) in the intercostal arch region distal of the middle line, approximately 2 cm to the right and to the left, was chosen as the place of application of the electrode.

4b—the lower part of the rectus abdominis muscle (m.rectus abdominis). innervation (Th_{V}-Th_{XII}, L_{I}), the lower abdomen approximately on the conditional line drawn between the upper frontal iliac spines (spina liaca anterioe superior) of the pelvic bones, approximately 1.5 cm to the right and to the left, was chosen as the place of application of the electrode.

X-XXI—as the place of application of the heating element, the place of the cervical and lumbar spine (C_{V}-L_{I}) was chosen, as well as the intervertebral symphysis (symphysis intervertebtralis) within the area, articulation joints (articulationes zygapophysiales), and the longitudinal yellow ligaments (ligg. longitudinales et flava) adjacent to them.

The adductor muscles of the hip (m.adductor longus femorale) (m.adductor magnus) are the antagonist muscles of the agonist muscles of the gluteus maximus muscle (m.gluteus medius) and the tensor muscle of fascia lata (m.tensor fasciae latae) and other muscles involved in the hip abduction, which, as a result of increased tonus, leads to the emergence of the opisthotonic posture—the set stimulation value was the maximum possible for the age and the muscle—60 μs, and a sedative thermal effect with a heating element by the temperature of 42° C. was added.

6a—as the place of application of the electrode, the place of proximal attachment of the long adductor of the thigh (m.adductor longus femorale) innervation (LII-LIII) and the great adductor muscle (m.adductor magnus), innervation (LII-LV) on the medial surface of the thigh 2 cm distal of the inguinal fold, was chosen.

6b—as the place of application of the electrode, the place of distal attachment of the great adductor muscle of the thigh (m.adductor magnus), innervation (LII-LV) on the medial surface of the thigh 2 cm proximal of the epicondylus of the femoral bone, was chosen.

Va—as the place of application of the heating element the following was chosen: the tendon of the quadriceps muscle of the thigh (m.quadriceps femoris) above the kneecap (patella), the tendon of the tailor's muscle of the thigh (m.sartorius); joint capsule (capsula articularis), the subcutaneous infrapatellar bursa (bursa subcutanea prepatellaris); the kneecap bursa (bursa suprapatellaris); the deep infrapatellar bursa (bursa infrapatellaris profunda); the subcutaneous infrapatellar bursa (bursa subcutanea infrapatellaris); the subcutaneous bursa of the tibial tuberosity (bursa subcutanea tuberositas tibiea); the subtendinous prepatellar bursa (bursa subtendinea prepatellaris); the anserine bursa (bursa anserina); joint capsule (capsula articularis).

The flexor of the foot—the triceps muscle of the calf (m.triceps surae) is the antagonist muscle of agonist muscles of the tibial muscle (m.tibialis anterior) and the long extensor of the fingers (m.extensor digitorum longus)—the set stimulation value was the maximum possible for the age and the muscle—60 μs, and a sedative thermal effect with a heating element by the temperature of 42° C. was added.

8c—the place of application of the electrode is the place of the proximal attachment of the triceps muscle of the calf (m.triceps surae), innervation (LIV-SII)

XXIIa—as the place of application of the heating element, the following was chosen: the triceps muscle of the thigh (caput longum m.biceps femori), the semitendinosus muscle (m. semitendinosus), the semimembranosus muscle (m. semimembranosus), the great adductor muscle (m.abductor magnus), the place of proximal attachment of the triceps muscle of the calf (m.triceps surae) on the back of the lower leg is 3 cm distal of the popliteal space.

8d—as the place of application of the electrode, the place of transition of the myogaster of the triceps muscle of the calf (m.triceps surae) to the Achilles tendon (tendo calcaneus (Achillis)), innervation (LIV-SI) was chosen, determined by palpation.

VIIa—as the place of the application of the heating element, the following was chosen: the place of the medial surface of the ankle joint (articulatio tibiocruralis) and the medial (deltoid) ligament (lig.mediale (deltoideum)) adjacent to it; the tibial-navicular ligament (lig.tibionavicularis); the anterior and posterior talus-tibial ligament (lig.tibiotalaris anterius et posterius); the anterior and posterior tibial-fibular ligament (lig.tibiofibulare anterius et posterius); the articular surface of the ankle (facies articularis maleoli tibiae); joint capsule (capsula articularis).

Muscle stimulation was carried out for at least 60 minutes a day by gradual calibration, alternating the active work of the patient in a stimulating suit, and passive loading through body position that allowed us to track changes in the patient's condition promptly.

After several sessions, the patient was able to bring his head to the position along the middle line and hold it by himself, which immediately affected the quality and quantity of food intake. Reducing the action of the asymmetric cervical tonic reflex allowed the patient to carry out his own developmental activity through the exploration of himself—he is now able to touch his face with his hand, feel it.

The number, intensity and duration of dystonic attacks decreased significantly. The duration and quality of night sleep has improved.

Arbitrary, isolated and precise movements of the hands appeared, which allows the patient to organize training, playing and cognitive activities in his own interests. Currently, the patient can independently bring his hands to the midline of the body and control their position to practice or rest at his own will or as the situation requires.

### Example 4

The patient is an 8 year old girl. Cerebral palsy, spastic diplegia.

Triple bending is complicated by a failed Achilles tendon tenotomy conducted in 2012. With spasmedity of the iliopsoas muscle and flexors of the knee joints, dorsal extension of the foot is observed. There are profound spasmedity of the adductors of the thigh muscles and the stiffness of the knee joints, the knees are fully extended only with external influence.

Spasmedity in the hands is insignificant. The shoulders are brought forward, at least there are difficulties with raising of both of the arms.

The position of the head is along the midline, the chin is in neutral position.

Uneven emotional background—the patient is permanently in a state of increased anxiety, intense muscle stimulation is impeded by hypersensitivity to the effects of this kind.

The segments of the spinal cord and agonist/antagonist muscle pairs were acted on simultaneously.

For stimulation, the following antagonist muscles were chosen to induce relaxation in spasmed agonist muscles.

Sternocleidomastoid (in. sternocleidomastoideus)—since re-bending is noticed only in the upper back, the specialist decided to create a sedative heat effect on the cervical spine with a heating element by the temperature of 42° C.

X-XI—the place of application of the heating element was dictated by the region of the cervical spine (C_{V}-C_{VII}).

Triceps (m.triceps brachii) is an antagonist muscle of agonist muscle of the biceps muscle of the arm (m.biceps brachii)—due to a slight spasmedity of the muscles of the upper limbs, the minimum stimulation value of 35 μs was set, and also a sedative heat effect with a heating element by the temperature of 42° C. was added.

1c—as the place of application of the electrode, the place of passage of the long head of the triceps muscle of the arm (caput longum m.triceps brachii) was chosen; innervation (C_{V}-C_{VIII}) of palpation-defined depression on the dorsal side of the humerus between the muscles of the posterior bundles of the deltoid muscle (m.deltoideus), the teres major muscle (m.teres major).

IXa—as the place of application of the heating element, the place of attachment of the myogaster of the biceps of the arm (venter m.biceps brachii) to the tuberosity of the radial bone (tuberositas radii) was chosen, as well as the humeroradial and humeroulnar joints (articulatio humeroradialis, articulatio humero-radialis) and the adjacent collateral radial ligament (lig.collaterale radiale), the annular radial ligament (lig.anulare radii), the square ligament (lig.quadratum); the tendon of the biceps muscle of the arm (tendo m.brachii (caput breve)); joint capsule (capsula articularis).

1 d—as the place of application of the electrode, the tendon of the triceps muscle of the arm (tendo m.triceps brachii) innervation (CV-CVII) was chosen, about 2 cm proximal of the olecranon process (olecranon).

IXb—as the place of application of the heating element, the place of attachment of the tendon of the triceps muscle of the arm (tendo m.triceps brachii) to the ulnar process of the ulna (olecranon ulnaris) was chosen, as well as the surface of the pomelolecteal joint (articulatio humeroulnaris) and the adjacent ulnar collateral ligament (lig. collaterale ulnare), the radial collateral ligament (lig.collaterale radiale); the intratendinous bursa (bursa intratendinea olecrani); the subcutaneous bursa of the olecranon (bursa subcutanea olecrani); the tendon of the triceps muscle of the arm (tendo m.triceps brachii); joint capsule (capsula articularis).

The carpi radialis and ulnar wrist extensors (m.extensor carpi ulnaris) (m.extensor carpi radialis) are antagonist muscles of agonist muscles of the ulnar wrist flexor (m.flexor carpi ulnaris), the radial wrist flexor (m.flexor carpi radialis), and the superficial digital flexor (m.digitorum superfacialis)—due to a slight spasmedity of the muscles of the upper limbs, the minimum stimulation value of 35 μs was set, and also a sedative heat effect with a heating element by the temperature of 42° C. was added.

3c—as the place of attachment of the electrode, the place of attachment of the myogaster of the ulnar extensor muscle of wrist (m.extensor carpi ulnaris) innervation (C_{VI}-C_{VIII}) was chosen, as well as the radial extensor muscle of the wrist (m.extensor carpi radialis) innervation (C_{V}-C_{VII}), and extensor muscles of the fingers (m.extensor digitorum) innervation (C_{VI}-C_{VIII}), approximately 2 cm distal of the ulnar process (olecranon).

IXb—as the place of application of the heating element, the place of attachment of the myogaster of the ulnar extensor muscle of wrist (m.extensor carpi ulnaris), the radial extensor muscle of the wrist (m.extensor carpi radialis), and extensor muscles of the fingers (m.extensor digitorum) to the ulnar process of the ulna (olecranon ulnaris) and the lateral elbow was chosen, as well as the surface of the pomelolecteal joint (articulatio humeroulnaris) and the adjacent ulnar collateral ligament (lig. collaterale ulnare), the radial collateral ligament (lig.collaterale radiale); the intratendinous bursa (bursa intratendinea olecrani); joint capsules (capsula articularis).

3d—the place located just above the wrist at the transition of the myogaster to the tendon part of the elbow wrist extensor (m.extensor carpi ulnaris) innervation (CVI-CVIII), radial wrist extensor (m.extensor carpi radialis) innervation (C_{V}-C_{VII}), and extensor muscles of the fingers (m.extensor digitorum) innervation (CVI-CVIII) was chosen as the place of application of the electrode, approximately 1-1.5 cm proximal of the radiocarpal joint between the ulnar and radial bones.

Ib—as the places of application of the heating element, the following were chosen: the back of the wrist joint (articulatio radiocarpea) and the distal radioulnar joint (articulatio radioulnaric distalis), and the adjoining back radiocarpal ligament (lig.radiocarpeum dorsale), and collateral radius and ulna carpal ligament (ligg. collaterale carpi radiale, collaterale carpi ulnare), tendons of the elbow extensor of the wrist (m.extensor carpi ulnaris), the radial extensor of the wrist (in.extensor carpi radialis), finger extensor (in.extensor digitorum); joint capsules (capsula articularis); as well as the tendon sheaths of the extensors of the fingers and the little finger (vagina tendinum mm extensor digitorum et extensoris indicis), the radial tendon of the wrist (vagina tendium mm extensorum carpi radialium), the ulnar tendon of the wrist (vagina tendinis mm extensor carpi ulnaris), the flexor tendon of the long finger of the hand (vagina tendinis m.extensoris policis longi).

The longissimus dorsi (m.longissimus) and the iliocostalis muscle (m.iliocostalis) are antagonist muscles of the agonist muscle of the rectus abdominis muscle (m.rectus abdominis)—due to the high anxiety and constant flexor spasmedity, the stimulation value was set to the maximum allowed for the age—40 μs, and a sedative thermal effect with a heating element by the temperature of 42° C. was added.

4c—as the place of imposition of the electrode, the following was chosen: the rib part of the longissimus dorsi (m.longissimus), the iliocostalis muscle (m.iliocostalis), innervation (Th_{I}-L_{I}), paravertebrally, approximately at the level of the fourth thoracic vertebra.

4d—as the place of imposition of the electrode, the following was chosen: the iliac parts of the longissimus dorsi (m.longissimus) and the iliocostalis muscle (m.iliocostalis). innervation (Th_{I}-L_{I}), paravertebrally, approximately at the level of the fourth thoracic vertebra.

X-XXI—as the place of application of the heating element, the place of the cervical and lumbar spine (C_{V}-L_{I}) was chosen, as well as the intervertebral symphysis (symphysis intervertebtralis) within the area, articulation joints (articulationes zygapophysiales), and the longitudinal yellow ligaments (ligg. longitudinales et flava) adjacent to them.

Tailor's muscles (m.sartorius), antagonists muscles of the agonist muscles of the biceps muscle of the thigh (caput longum m.biceps femori), of the semiburnal muscles (in.semitendinosus), the semimembranosus (m.semimembranosus), and the great adductor muscle (m.abductor magnus)—due to the high anxiety and the increased flexor spasmedity of the iliopsoas muscle and knee flexor muscles, the specialist considered it possible to set a moderate stimulation value of 60 μs so that the patient felt more comfortable and more confident, and also a sedative thermal effect with a heating element by the temperature of 42° C. was added.

7a—as the place of attachment of the electrode the following was chosen: the place of attachment of the tailor's muscle of the thigh (m.sartorius) innervation (L_{H}-L_{III}); quadriceps muscle of the thigh (m.quadriceps femoris, m.rectus femoris) innervation (L_{III}-L_{IV}) lateral part of the thigh 2 cm medial of the greater trochanter (trochanter major)

IV—as the place of attachment of the electrode the following was chosen: the proximal part of the quadriceps muscle of thigh (m.quadriceps femoris), the proximal part of the tailor's muscle of thigh (m.sartorius), the gluteus maximus muscle (m.gluteus maximus); the gluteus medius muscle (m.gluteus medius); the gluteus minimus muscle (m.gluteus minimus); the square muscle of thigh (m.qudratus femoris); tensor muscle of fascia lata (m.tensor fasciae latae); as well as the surface of the hip joint (articulatio coxae) and the transverse bundle of the acetabulum adjacent to it (lig.transversum acetabuli); iliofemoral ligament (lig.iliofemorale); pubofemoral ligament (lig.pubofemorale); sciatic-femoral ligament (lig.ischiofemorale); circular zone (zona orbicularis); ligament of the femoral head (lig.capitis femoris); the tendon of the biceps of the arm (tendo m.brahii (caput breve)); bursa of the gluteus maximus muscle (bursa trochanterica in. glutei maximi); the intermuscular bursa of gluteus muscles (bursae intermusculares mm gluteorum); ischial bursa of gluteus maximus (bursa ischiadica m.glutei maximi); trochanteric bursa of the gluteus maximus muscle (burse trochantericae m.glutei medii); trochanteric bursa of the gluteus minimus muscle (bursa trochanterica m.glutei minimi); joint capsule (capsula articularis).

7b—as the place of application of the electrode, the place of transition of the distal part of the myogaster of the quadriceps muscle of the thigh (m.quadriceps femoris) into the tendon, innervation (L_{III}-L_{IV}) on the medial surface of the thigh 2 cm proximal of the medial epicondylus of the femoral bone (edicondylus medialis).

Va—as the place of application of the heating element the following was chosen: the tendon of the quadriceps muscle of the thigh (m.quadriceps femoris) above the kneecap (patella), the tendon of the tailor's muscle of the thigh (m.sartorius); as well as the upper surface of the knee joint (articulatio genus); the subcutaneous infrapatellar bursa (bursa subcutanea prepatellaris); kneecap bursa (bursa suprapatellaris); the deep infrapatellar bursa (bursa infrapatellaris profunda); the subcutaneous infrapatellar bursa (bursa subcutanea infrapatellaris); the subcutaneous bursa of the tibial tuberosity (bursa subcutanea tuberositas tibiea); the subtendinous prepatellar bursa (bursa subtendinea prepatellaris); the anserine bursa (bursa anserina); joint capsule (capsula articularis).

The triceps muscle of the calf (m.triceps surae) is the antagonist muscle of the agonist muscles of the tibial muscle (m.tibialis anterior) and the long extensor of the fingers (m.extensor digitorum longus)—after the unsuccessfully carried out tenotomy of Achilles tendon, significant dorsal flexion of the foot is observed. To weaken the effect of surgical intervention, it was decided to set the maximum stimulation value to 60 μs, and also a sedative thermal effect with a heating element by the temperature of 42° C. was added.

8c—the place of application of the electrode is the place of the proximal attachment of the triceps muscle of the calf (m.triceps surae), innervation (LIV-SII)

XXIIa—as the place of application of the heating element, the following was chosen: the triceps muscle of the thigh (caput longum in.biceps femori), the semitendinosus muscle (m. semitendinosus), the semimembranosus muscle (m. semimembranosus), the great adductor muscle (m.abductor magnus), the place of proximal attachment of the triceps muscle of the calf (m.triceps surae) on the back of the lower leg is 3 cm distal of the popliteal space.

8d—as the place of application of the electrode, the place of transition of the myogaster of the triceps muscle of the calf (m.triceps surae) to the Achilles tendon (tendo calcaneus (Achillis)), innervation (LIV-SI) was chosen, determined by palpation.

VIIa—as the place of the application of the heating element, the following was chosen: the place of the medial surface of the ankle joint (articulatio tibiocruralis) and the medial (deltoid) ligament (lig.mediale (deltoideum)) adjacent to it; the tibial-navicular ligament (lig.tibionavicularis); the anterior and posterior talus-tibial ligament (lig.tibiotalaris anterius et posterius); the anterior and posterior tibial-fibular ligament (lig.tibiofibulare anterius et posterius); the articular surface of the ankle (facies articularis maleoli tibiae); joint capsule (capsula articularis).

Muscle stimulation was carried out by gradual calibration, alternating the active work of the patient in a stimulating suit, and passive loading through body position that allowed us to track changes in the patient's condition promptly. Stimulation was carried out uniformly on both sides.

After the first two days of stimulation, the patient experienced general fatigue in the stimulated muscles and an increase in psycho-motor agitation. Three days later, the emotional background returned to normal, spasmedity in the feet decreased, and the tension in the adductor thigh muscles eased. The patient was able to perform side-steps, the foot supportability increased, the patient began to put both legs on the floor evenly.

The tonic bending of the body decreased noticeably.

Night sleep improved, fine motor skills in the hands appeared. As a result, the patient developed a desire for self-occupation and cognitive development. The fear of failure and anxiety decreased.

### INDUSTRIAL APPLICABILITY

To implement the invention, well-known materials and equipment are used, which determines, in the opinion of the applicant, conformity of the claimed invention to the patentability criterion of “Industrial Applicability” (“IA”).

TABLE 1
Areas of exposure of the agonist/antagonist muscle pairs

| Agonist muscle | | Antagonist muscle | |
|---|---|---|---|
| Designation 1 | Description 2 | Designation 3 | Description 4 |
| 1a | the place of imposition of the | 1c | the place of application of the |
| | electrode is the proximal part of | | electrode is the place of passage |
| | the myogaster of the biceps | | of the long head of the triceps |
| | muscle of one of the arm | | muscle of the arm (caput |
| | (venter) m.biceps brachii) at the | | longum m.triceps brachii) |
| | place of its transition into the | | innervation (C_{V}-C_{VIII}) |
| | tendon (tendo m.biceps brachii) | | |
| | innervation (C_{V}-C_{VII}) | | |
| Ia | the place of application of the | IXa | the place of application of the |
| | heating element is localized by | | heating element is localized by |
| | the point of attachment of the | | the place of attachment of the |
| | short head of the biceps muscle | | myogaster of the biceps of the |
| | of the arm (caput breve | | arm (venter m.biceps brachii) to |
| | m.biceps brachii) to the coracoid | | the tuberosity of the radial bone |
| | process of the scapula | | (tuberositas radii), as well as the |
| | (processus coracoideus scapula), | | humeroradial and humeroulnar |
| | as well as the front surface of | | joints (articulatio |
| | the shoulder joint (articulation | | humeroradialis, articulatio |
| | humeri) and the adjacent | | humero-radialis) and the |
| | glenohumeral, coracoid- | | adjacent collateral radial |
| | acromial and coraco-humeral | | ligament (lig.collaterale |
| | ligaments (ligg.glenohurale | | radiale), the annular radial |
| | superius, mediale, inferius; | | ligament (lig.anulare radii), the |
| | lig.coracoacromeale; | | square ligament |
| | lig.coracohumorale); the tendon | | (lig.quadratum); the tendon of |
| | of the biceps muscle of the arm | | the biceps muscle of the arm |
| | (tendo m.brahii (caput breve)); | | (tendo m.brachii (caput |
| | joint capsule (capsula articularis). | | breve)); joint capsule (capsula |
| | | | articularis). |
| 1b | the place of imposition of the | 1d | the place of application of the |
| | electrode is the distal part of the | | electrode is the tendon of the |
| | myogaster of the biceps muscle | | triceps muscle of the arm (tendo |
| | of one of the arms (venter) | | m.triceps brachii) innervation |
| | m.biceps brachii) innervation (C_{V}-C_{VII}) | | (C_{V}-C_{VII}). |
| Ib | the place of application of the | IXb | the place of application of the |
| | heating element is localized by | | heating element is localized by |
| | the point of attachment of the | | the place of attachment of the |
| | long head of the biceps muscle | | tendon of the triceps muscle of |
| | of the arm (caput longum | | the arm (tendo m.triceps |
| | m.triceps brachii) to the | | brachii) to the ulnar process of |
| | articular tubercle of the scapula | | the ulna (olecranon ulnaris), as |
| | (tuberculum infraglenoidale | | well as the surface of the |
| | angulus lateralis scapule), as | | pomelol ecteal joint (articulatio |
| | well as the posterior surface of | | humeroulnaris) and the adjacent |
| | the shoulder joint (articulation | | ulnar collateral ligament (lig. |
| | humeri) and the transverse and | | collaterale ulnare), the radial |
| | beak-humeral ligaments | | collateral ligament |
| | adjacent to it (lig.transversum | | (lig.collaterale radiale); the |
| | scapule; | | intratendinous bursa (bursa |
| | lig.coracohumorale); tendon of | | intratendinea olecrani); the |
| | the long head of the triceps | | subcutaneous bursa of the |
| | muscle of the arm (caput | | olecranon (bursa subcutanea |
| | longum m.triceps brachii); joint | | olecrani); the tendon of the |
| | capsule (capsula articularis), | | triceps muscle of the arm (tendo |
| | subacromial bursa (bursa | | m.triceps brachii); joint capsule |
| | subacromialis). | | (capsula articularis). |
| 2a | the place of application of the | 2a | the place of application of the |
| | electrode is the place of the distal | | electrode is the place of the |
| | part of the myogaster of the deltoid | | distal part of the myogaster of |
| | muscle (venter m.deltoideus) and | | the deltoid muscle (venter |
| | the attachment of the pectoralis | | m.deltoideus) and the |
| | major muscle to the humerus | | attachment of the pectoralis |
| | (mm.pectoralis major), innervation | | major muscle to the humerus |
| | (C_{V}-C_{VI}) | | (mm.pectoralis major), |
| | | | innervation (C_{V}-C_{VI}) |
| Ia | the place of application of the | Ia | the place of application of the heating |
| | heating element is localized by | | element is localized by the point of |
| | the point of attachment of the | | attachment of the short head of the |
| | short head of the biceps muscle | | biceps muscle of the arm |
| | of the arm (caput breve | | (caput breve m.biceps brachii) to the |
| | m.biceps brachii) to the | | coracoid process of the scapula |
| | coracoid process of the scapula | | (processus coracoideus scapula), |
| | (processus coracoideus scapula), | | as well as the attachment of the deltoid |
| | as well as the attachment of the | | muscle (venter m.deltoideus) |
| | deltoid muscle (venter | | and the pectoralis major muscle |
| | m.deltoideus) and the pectoralis | | (mm.pectoralis major) to the crest of |
| | major muscle (mm.pectoralis | | the large tubercle (crista tuberculi |
| | major) to the crest of the large | | majoris), and the front surface of the |
| | tubercle (crista tuberculi | | shoulder joint (articulation humeri) |
| | majoris), and the front surface | | and the adjacent glenohumeral, |
| | of the shoulder joint | | coracoid-acromial and coraco-humeral |
| | (articulation humeri) and the | | ligaments (ligg.glenohurale superius, |
| | adjacent glenohumeral, | | mediale, inferius). |
| | coracoid-acromial and coraco-humeral | | |
| | ligaments (ligg.glenohurale superius, | | |
| | mediale, inferius; lig.coracoacromeale; | | |
| | lig.coracohumorale); the tendon of the | | |
| | biceps muscle of the arm (tendo m.brahii | | |
| | (caput breve)); joint capsule (capsula | | |
| | articularis), subdeltoid bursa (bursa | | |
| | subdeltoidea). | | |
| 2b | the place of imposition of the | 2d | the place of application of the |
| | electrode is the proximal part of | | electrode is the distal part of the |
| | the myogaster of the pectoralis | | myogaster of the deltoid muscle |
| | major muscle (mm.pectoralis | | (venter m.deltoideus) and |
| | major) and the anterior part of | | the distal part of the myogaster of the |
| | the myogaster of the deltoid | | circular muscle (mm.teres major et |
| | muscle (venter m.deltoideus | | minor) innervation (C_{V}-C_{VI}) |
| | anterior) innervation (C_{V}-C_{VI}) | | |
| III | [symbol] | Ib | the place of application of the heating |
| | [symbol] | | element is localized by the point of |
| | [symbol] | | attachment of the long head of the |
| | [symbol] | | triceps muscle of the arm (caput |
| | [symbol] | | longum m.triceps brachii) to the |
| | (mm.pectoralis major) κ | | articular tubercle of the scapula |
| | [symbol] | | (tuberculum infraglenoidale angulus |
| | [symbol] | | lateralis scapule), as well as the |
| | [symbol] | | posterior surface of the shoulder |
| | [symbol] | | joint (articulation humeri) and the |
| | [symbol] | | transverse and beak-humeral |
| | [symbol] [symbol] | | ligaments adjacent to it |
| | [symbol] | | (lig.transversum scapule; |
| | [symbol] | | lig.coracohumorale); tendon of the |
| | [symbol] | | long head of the triceps muscle of |
| | (lig.sternoclaviculare; lig.costaclaviculare, | | the arm (caput longum m.triceps |
| | [symbol] | | brachii); joint capsule (capsula |
| | [symbol] | | articularis), subacromial bursa |
| | | | (bursa subacromialis), the crest of |
| | | | the lesser tubercle of humeri of the |
| | | | humerus (crista tuberculi minor). |
| 3a | [symbol] | 3c | the place of application of the |
| | [symbol] | | electrode is the place of attachment |
| | [symbol] | | of the myogaster of the elbow wrist |
| | [symbol] | | extensor (m.extensor carpi ulnaris) |
| | [symbol] | | innervation (C_{VI}-C_{VIII}), radial |
| | [symbol] | | wrist extensor (m.extensor carpi |
| | [symbol] | | radialis) innervation (C_{V}-C_{VII}), |
| | [symbol] | | and extensor muscles of the fingers |
| | [symbol] | | (m.extensor digitorum) innervation |
| | [symbol] | | (C_{VI}-C_{VIII}) |
| | [symbol] | | |
| | [symbol] | | |
| | m.flexor digitorum profundus) | | |
| | [symbol] | | |
| IXa | the place of application of the heating | IXb | the place of application of the |
| | element is localized by the attachment | | heating element is localized by |
| | point of the myogaster of the elbow | | the place of attachment of the |
| | flexor of the wrist (m.flexor carpi | | myogaster of the ulnar extensor |
| | ulnaris), the radial flexor of the wrist | | muscle of wrist (m.extensor |
| | (m. flex or carpi radialis), the surface | | carpi ulnaris), the radial extensor |
| | flexor of the fingers (m.digitorum | | muscle of the wrist (m.extensor |
| | superfacialis), the myogaster of the | | carpi radialis), and extensor |
| | deep flexor of the fingers (venter | | muscles of the fingers |
| | m.flexor digitorum profundus) to the | | (m.extensor digitorum) to the |
| | tuberosity of the radial bone | | ulnar process of the ulna |
| | (tuberositas radii) and the medial | | (olecranon ulnaris) and the |
| | epicondylee (epicondylus medialis), | | lateral elbow was chosen, as |
| | as well as the surface of the humero- | | well as the surface of the |
| | veltic joint (articulatio humeroulnaris) | | pomelolecteal joint (articulatio |
| | and the adjacent ulnar collateral | | humeroulnaris) and the adjacent |
| | ligament (lig.collaterale ulnare), the | | ulnar collateral ligament (lig. |
| | radial collateral ligament (collaterale | | collaterale ulnare), the radial |
| | radiale); the intratendinous bursa | | collateral ligament |
| | (bursa intratendinea olecrani); the | | (lig. collaterale radiale); the |
| | tendon of the triceps muscle of the | | intratendinous bursa (bursa |
| | arm (tendo m.triceps brachii); joint | | intratendinea olecrani); joint |
| | capsule (capsula articularis). | | capsules (capsula articularis). |
| 3b | the place of application of the electrode | 3d | the place of application of the |
| | is the place located just above the | | electrode is the place located just |
| | wrist at the level of the myogaster | | above the wrist at the transition |
| | transition into the tendon part of the | | of the myogaster to the tendon |
| | elbow flexor of the wrist (m.flexor | | part of the elbow wrist extensor |
| | carpi ulnaris) innervation (C_{VII}-C_{VIII}), | | (m. extensor carpi ulnaris) |
| | radial flexor of the wrist | | innervation (C_{VI}-C_{VIII}), radial |
| | (m.flexor carpi radialis) innervation | | wrist extensor (m.extensor carpi |
| | (C_{VI}-C_{VII}), surface flexor of the | | radialis) innervation(C_{V}-C_{VII}), |
| | fingers (m.digitorum superfacialis) | | and extensor muscles of the |
| | innervation (C_{VII}-Th_{I}), myogaster | | fingers (m.extensor digitorum) |
| | of the deep flexor of the fingers | | innervation (C_{VI}-C_{VIII}) |
| | (venter m.flexor digitorum | | |
| | profundus) innervation (C_{VI}-Th_{I}) | | |
| IIa | the place of application of the heating | Ib | the place of application of the |
| | element is localized by the place of | | heating element is localized by |
| | the anterior surface of the wrist joint | | the back of the wrist joint |
| | (articulatio radiocarpea) and the distal | | (articulatio radiocarpea) and the |
| | radioulnar joint (articulatio | | distal radioulnar joint (articulatio |
| | radioulnaric distalis), and the adjacent | | radioulnaric distal is), and the |
| | palmar and elbow wrist ligaments | | adjoining back radiocarpal |
| | (the ligg.radiocarpeum palmare) | | ligament (lig.radiocarpeum |
| | tendons of the elbow flexor of the | | dorsale), and collateral radius |
| | wrist (m.flexor carpi ulnaris), radial | | and ulna carpal ligament (ligg. |
| | flexor of the wrist (m. flex or carpi | | collaterale carpi radiale, |
| | radialis), surface flexor of the fingers | | collaterale carpi ulnare), tendons |
| | (m.digitorum superfacial is), abdomen | | of the elbow extensor of the |
| | of the deep flexor of the fingers | | wrist (m.extensor carpi ulnaris), |
| | (venter m. flexor digitor profundus); | | the radial extensor of the wrist |
| | joint capsules (capsula articularis); as | | (m. extensor carpi radial is), |
| | well as the sheaths of the common | | finger extensor (m. extensor |
| | flexor of the fingers (vagina | | digitorum); joint capsules |
| | communis mm.flexorum), the tendon | | (capsula articularis); as well as |
| | of the long flexor of the thumb | | the tendon sheaths of the |
| | (vagina tendinis m.flexoris policis longi). | | extensors of the fingers and the |
| | | | little finger (vagina tendinum |
| | | | mm. extensor digitorum et |
| | | | extensoris indicis), the radial |
| | | | tendon of the wrist (vagina |
| | | | tendium mm. extensorum carpi |
| | | | radialium), the ulnar tendon of |
| | | | the wrist (vagina tendinis mm. |
| | | | extensor carpi ulnaris), the |
| | | | flexor tendon of the long finger |
| | | | of the hand (vagina tendinis |
| | | | m.extensoris policis longi). |
| 5a | the place of application of the electrode | 5c | the place of application of the |
| | is the place of attachment of the | | electrode is the place of |
| | tailor's muscle of the thigh | | attachment of the gluteus |
| | (m.sartorius), innervation (L_{II}-L_{III}) | | maximus muscle (m.gluteus |
| | | | maximus); innervation (L_{V}-S_{I}) |
| IV | the place of application of the heating | XXI | the place of application of the |
| | element is localized by the attachment | | heating element is localized by |
| | point of the proximal part of the | | the area of the lumbar and |
| | quadriceps muscle of thigh | | sacral spine (L1-S5), as well |
| | (m.quadriceps femoris), the proximal | | as the intervertebral symphysis |
| | part of the tailor's muscle of thigh | | (symphysis intervertebtralis) |
| | (m.sartorius), the gluteus maximus | | within the area, and the |
| | muscle (m.gluteus maximus); the | | longitudinal yellow ligaments |
| | gluteus medius muscle (m.gluteus | | (ligg. longitudinales et flava) |
| | medius); the gluteus minimus muscle | | adjacent to them. |
| | (m.gluteus minimus); the square | | |
| | muscle of thigh (m.qudratus femoris); | | |
| | tensor muscle of fascia lata (m.tensor | | |
| | fasciae latae); as well as the surface of | | |
| | the hip joint (articulatio coxae) and the | | |
| | transverse bundle of the acetabulum | | |
| | adjacent to it (lig.transversum | | |
| | acetabuli); iliofemoral ligament | | |
| | (lig.iliofemorale); pubofemoral | | |
| | ligament (lig.pubofemorale); sciatic- | | |
| | femoral ligament (lig.ischiofemorale); | | |
| | circular zone (zona orbicularis); | | |
| | ligament of the femoral head | | |
| | (lig.capitis femoris); the tendon of the | | |
| | biceps of the arm (tendo m.brahii | | |
| | (caput breve)); bursa of the gluteus | | |
| | maximus muscle (bursa trochanterica | | |
| | m. glutei maximi); the intermuscular | | |
| | bursa of gluteus muscles (bursae | | |
| | intermusculares mm.gluteorum); | | |
| | ischial bursa of gluteus maximus | | |
| | (bursa ischiadica m.glutei maximi); | | |
| | trochanteric bursa of the gluteus | | |
| | maximus muscle (burse trochantericae | | |
| | m.glutei medii); trochanteric bursa of | | |
| | the gluteus minimus muscle (bursa | | |
| | trochanterica m.glutei minimi); joint | | |
| | capsule (capsula articularis). | | |
| 5b | the place of application of the | 5d | the place of application of the |
| | electrode is the place of transition of | | electrode is the place of distal |
| | the distal myogaster of attachment of | | attachment of the gluteus |
| | the tailor's muscle of the thigh | | maximus muscle (m.gluteus |
| | (m.sartorius) into the tendon, | | maximus) to the femoral bone. |
| | innervation (L_{II}-L_{III}) | | innervation (LV-SI) |
| Va | the place of application of the heating | IV | the place of attachment of the |
| | element is localized by the tendon of | | electrode is localized by the |
| | the quadriceps muscle of the thigh | | proximal part of the quadriceps |
| | (m.quadriceps femoris) above the | | muscle of thigh (m.quadriceps |
| | kneecap (patella), the tendon of the | | femoris), the proximal part of the |
| | tailor's muscle of the thigh | | tailor's muscle of thigh |
| | (m.sartorius); joint capsule (capsula | | (m.sartorius), the gluteus maximus |
| | articularis), the subcutaneous | | muscle (m.gluteus maximus); the |
| | infrapatellar bursa (bursa subcutanea | | gluteus medius muscle (m.gluteus |
| | prepatellaris); kneecap bursa (bursa | | medius); the gluteus minimus |
| | suprapatellaris); the deep infrapatellar | | muscle (m.gluteus minimus); the |
| | bursa (bursa infrapatellaris profunda); the | | square muscle of thigh (m.qudratus |
| | subcutaneous infrapatellar bursa | | femoris); tensor muscle of fascia |
| | (bursa subcutanea infrapatellaris); the | | lata (m.tensor fasciae latae); as well |
| | subcutaneous bursa of the tibial tuberosity | | as the surface of the hip joint |
| | (bursa subcutanea tuberositas tibiea); the | | (articulatio coxae) and the |
| | subtendinous prepatellar bursa (bursa | | transverse bundle of the acetabulum |
| | subtendinea prepatellaris); the anserine bursa | | adjacent to it (lig.transversum |
| | (bursa anserina). | | acetabuli); iliofemoral ligament |
| | | | (lig.iliofemorale); pubofemoral |
| | | | ligament (lig.pubofemorale); |
| | | | sciatic-femoral ligament |
| | | | (lig.ischiofemorale); circular zone |
| | | | (zona orbicularis); ligament of the |
| | | | femoral head (lig.capitis femoris); |
| | | | the tendon of the biceps of the arm |
| | | | (tendo m.brahii (caput breve)); |
| | | | bursa of the gluteus maximus |
| | | | muscle (bursa trochanterica m. |
| | | | glutei maximi); the intermuscular |
| | | | bursa of gluteus muscles (bursae |
| | | | intermusculares mm.gluteorum); |
| | | | ischial bursa of gluteus maximus |
| | | | (bursa ischiadica m.glutei |
| | | | maximi); trochanteric bursa of |
| | | | the gluteus maximus muscle |
| | | | (burse trochantericae m.glutei |
| | | | medii); trochanteric bursa of the |
| | | | gluteus minimus muscle (bursa |
| | | | trochanterica m.glutei minimi); |
| | | | joint capsule (capsula articularis). |
| 6a | the place of application of the | 6c | the place of application of the |
| | electrode is the place of proximal | | electrode is the place of the |
| | attachment of the long adductor of | | proximal attachment of the gluteus |
| | the thigh (m.adductor longus | | medius muscle (m.gluteus medius), |
| | femorale) innervation (L_{II}-L_{III}) and | | innervation (L_{IV}- S_{I}) and the tensor |
| | the great adductor muscle (m.adductor | | muscle of fascia lata (m.tensor fasciae |
| | magnus), innervation (L_{II}-L_{V}) | | latae). innervation(L_{IV}-S_{I}) |
| 6b | the place of application of the | IV | the place of application of the heating |
| | electrode is the place of distal | | element is localized by the attachment |
| | attachment of the great adductor | | point of the proximal part of the |
| | muscle of the thigh (m.adductor | | quadriceps muscle of thigh |
| | magnus) innervation (L_{II}-L_{V}) | | (m.quadriceps femoris), the proximal |
| | | | part of the tailor's muscle of thigh |
| | | | (m.sartorius), the gluteus maximus |
| | | | muscle (m.gluteus maximus); the |
| | | | gluteus medius muscle (m.gluteus |
| | | | medius); the gluteus minimus muscle |
| | | | (m.gluteus minimus); the square |
| | | | muscle of thigh (m.qudratus femoris); |
| | | | tensor muscle of fascia lata (m.tensor |
| | | | fasciae latae); as well as the surface of |
| | | | the hip joint (articulatio coxae) and the |
| | | | transverse bundle of the acetabulum |
| | | | adjacent to it (lig.transversum |
| | | | acetabuli); iliofemoral ligament |
| | | | (lig.iliofemorale); pubofemoral |
| | | | ligament (lig.pubofemorale); sciatic- |
| | | | femoral ligament (lig.ischiofemorale); |
| | | | circular zone (zona orbicularis); |
| | | | ligament of the femoral head |
| | | | (lig.capitis femoris); the tendon of the |
| | | | biceps of the arm (tendo m.brahii |
| | | | (caput breve)); bursa of the gluteus |
| | | | maximus muscle (bursa trochanterica |
| | | | m. glutei maximi); the intermuscular |
| | | | bursa of gluteus muscles (bursae |
| | | | intermusculares); ischial bursa of |
| | | | gluteus maximus (bursa ischiadica); |
| | | | trochanteric bursa of the gluteus |
| | | | maximus muscle (burse |
| | | | trochantericae); trochanteric |
| | | | bursa of the gluteus minimus muscle |
| | | | (bursa trochanterica); joint capsule. |
| Va | the place of application of the heating | 6d | the place of application ot the |
| | element is localized by the tendon of | | electrode is the place of the |
| | the quadriceps muscle of the thigh | | distal attachment of the gluteus |
| | (m.quadriceps femoris) above the | | medius muscle (m.gluteus |
| | kneecap (patella), the tendon of the | | medius), innervation (L_{IV}-S_{I}) |
| | tailor's muscle of the thigh | | and the transition of the |
| | (m.sartorius); joint capsule (capsula | | myogaster of the tensor muscle |
| | articularis), the subcutaneous | | of fascia lata (m.tensor fasciae latae) |
| | infrapatellar bursa (bursa subcutanea | | into the tendon, innervation (L_{IV}-S_{I}) |
| | prepatellaris); kneecap bursa (bursa | | |
| | suprapatellaris); the deep infrapatellar | | |
| | bursa (bursa infrapatellaris profunda); | | |
| | the subcutaneous infrapatellar bursa | | |
| | (bursa subcutanea infrapatellaris); the | | |
| | subcutaneous bursa of the tibial | | |
| | tuberosity (bursa subcutanea | | |
| | tuberositas tibiea); the subtendinous | | |
| | prepatellar bursa (bursa subtendinea | | |
| | prepatellaris); the anserine bursa (bursa | | |
| | anserina); joint capsule (capsula_ | | |
| | articularis). | | |
| 7a | the place of attachment of the | 7c | the place of application of the |
| | electrode is the place of attachment of | | electrode-is the place of |
| | the tailor's muscle of the thigh | | attachment of the biceps muscle |
| | (m.sartorius) innervation (L_{II}-L_{III}); | | of the thigh (caput longum |
| | quadriceps muscle of the thigh | | m.biceps femori), innervation |
| | (m. quadriceps femoris, m. rectus | | (L_{IV}-L_{V}, S_{I}), semitendinosus |
| | femoris) innervation (L_{III}-L_{IV}) | | muscle (m.semitendinosus) |
| | | | innervation (L_{IV}-L_{V}, S_{I}), |
| | | | semimembranosus muscle |
| | | | (m. semimembranosus) |
| | | | innervation (L_{IV}- L_{V}, S_{I}), great |
| | | | adductor muscle (m.abductor |
| | | | magnus) innervation (L_{IV}-L_{V}, S_{I}) |
| IV | the place of attachment of the | XXIIa | the place of application of the |
| | electrode is localized by the proximal | | heating element is localized by |
| | part of the quadriceps muscle of thigh | | the place of passage of the |
| | (m. quadriceps femoris), the proximal | | tendons of the biceps muscle of |
| | part of the tailor's muscle of thigh | | the thigh (caput longum m.biceps |
| | (m.sartorius), the gluteus maximus | | femori), the semitendinosus |
| | muscle (m.gluteus maximus); the | | muscle (m.semitendinosus), the |
| | gluteus medius muscle (m. gluteus | | semimembranosus muscle |
| | medius); the gluteus minimus muscle | | (m.semimembranosus), the great |
| | (m.gluteus minimus); the square | | adductor muscle (m.abductor magnus). |
| | muscle of thigh (m.qudratus femoris); | | |
| | tensor muscle of fascia lata (m.tensor | | |
| | fasciae latae); as well as the surface of | | |
| | the hip joint (articulatio coxae) and the | | |
| | transverse bundle of the acetabulum | | |
| | adjacent to it (lig.transversum | | |
| | acetabuli); iliofemoral ligament | | |
| | (lig.iliofemorale); pubofemoral | | |
| | ligament (lig.pubofemorale); sciatic- | | |
| | femoral ligament (lig.ischiofemorale); | | |
| | circular zone (zona orbicularis); | | |
| | ligament of the femoral head | | |
| | (lig.capitis femoris); the tendon of the | | |
| | biceps of the arm (tendo m.brahii | | |
| | (caput breve)); bursa of the gluteus | | |
| | maximus muscle (bursa trochanterica | | |
| | m. glutei maximi); the intermuscular | | |
| | bursa of gluteus muscles (bursae | | |
| | intermusculares mm.gluteorum); | | |
| | ischial bursa of gluteus maximus | | |
| | (bursa ischiadica m.glutei maximi); | | |
| | trochanteric bursa of the gluteus | | |
| | maximus muscle (burse trochantericae | | |
| | m.glutei medii); trochanteric bursa of | | |
| | the gluteus minimus muscle (bursa | | |
| | trochanterica m.glutei minimi); joint | | |
| | capsule (capsula articularis). | | |
| 7b | the place of application of the | 7d | the place of application of the |
| | electrode is the place of transition of | | electrode is the place of the |
| | the distal part of the myogasters of the | | proximal transition of the |
| | quadriceps muscle of the thigh | | myogaster into the tendon of the |
| | (m. quadriceps femoris) into the | | triceps muscle of the thigh (caput |
| | tendon, innervation (L_{III} - L_{IV}) | | longum m.biceps femori) |
| | | | innervation (L_{III}-L_{IV}), the |
| | | | semitendinousus muscle |
| | | | (m.semitendinosus) innervation |
| | | | (L_{III}-L_{IV}), the semimembranosus |
| | | | muscle (m. semimembranosus) |
| | | | innervation (L_{III}-L_{IV}), the great |
| | | | adductor muscle (m.abductor |
| | | | magnus) innervation (L_{III}-L_{IV}) |
| Va | the place of application of the heating | Vb | the place of application of the heating |
| | element is localized by the tendon of | | element is localized by the proximal |
| | the quadriceps muscle of the thigh | | transition of the myogaster into the |
| | (m.quadriceps femoris) above the | | tendon of the triceps muscle of the |
| | kneecap (patella), the tendon of the | | thigh (caput longum m.biceps femori), |
| | tailor's muscle of the thigh | | the semitendinosus muscle |
| | (m.sartorius); joint capsule (capsula | | (m.semitendinosus),the semimem- |
| | articularis), the subcutaneous | | branosus muscle (m. |
| | infrapatellar bursa (bursa subcutanea | | semimembranosus) the great |
| | prepatellaris); kneecap bursa (bursa | | adductor muscle (m.abductor |
| | suprapatellaris); the deep infrapatellar | | magnus) into the adductor |
| | bursa (bursa infrapatellaris profunda); | | tubercle of the femoral bone |
| | the subcutaneous infrapatellar bursa | | (tuberculum adductorium) and |
| | (bursa subcutanea infrapatellaris); the | | fascia of the posterior surface of |
| | subcutaneous bursa of the tibial | | the knee joint, as well as the |
| | tuberosity (bursa subcutanea | | posterior surface of the knee |
| | tuberositas tibiea); the subtendinous | | joint (articulatio genus) and the |
| | prepatellar bursa (bursa subtendinea | | adjacent oblique popliteal |
| | prepatel laris); the anserine bursa (bursa | | ligament (lig. popliteum |
| | anserina); joint capsule (capsula | | obliquum) and the arcuate popliteal |
| | articularis). | | ligament lig.popliteum arcuatum);_ |
| | | | the cruciate ligament of the knee |
| | | | (ligg.cruata genus); the transverse |
| | | | knee ligament (lig.transversum |
| | | | genus); the meniscofemoral |
| | | | ligament (ligg.meniscofemorale |
| | | | anterius et posterius); meniscus |
| | | | (meniscus medialis et lateralis); the |
| | | | semimembranous bursa (bursa |
| | | | semimembranosus); the lower the |
| | | | subcutaneous bursa of the biceps |
| | | | muscle of the thigh (bursa |
| | | | subtendinea m.bicipitis femoris |
| | | | inferior); joint capsule (capsula |
| | | | articularis). |
| 8a | the place of application of the | 8c | the place of application of the |
| | electrode is the place of the distal | | electrode is the place of the |
| | attachment of the anterior tibial | | proximal attachment of the |
| | muscle (m.tibialis anterior), | | triceps muscle of the calf |
| | innervation (L_{IV}-S_{I}) and the long | | (m.triceps surae), innervation |
| | extensor of the fingers (m.extensor | | (L_{IV}-S_{II}) |
| | digitorum longus) to the shin bone, | | |
| | innervation (L_{IV}-S_{I}) | | |
| VI | the place of application of the heating | XXIIaa | the place of application of the |
| | element is localized by the place of | | heating element is localized by |
| | proximal attachment of the anterior | | the place of transition of the |
| | tibial muscle (m.tibialis anterior) and | | triceps muscle of the thigh (caput |
| | the long extensor of the fingers | | longum m.biceps femori), the |
| | (m.extensor digitorum longus) to the | | semitendinosus muscle |
| | medial condylus of the shin bone | | (m.semitendinosus), the |
| | (condylus medialis); as well as the | | semimembranosus muscle |
| | lower surface of the knee joint | | (m.semimembranosus), the great |
| | (articulatio genus) and the adjacent | | adductor muscle (m. abductor |
| | tendons of the quadriceps muscle of | | magnus), the place of proximal |
| | the thigh (tendo m.quadriceps | | attachment of the triceps muscle |
| | femoris) below the kneecap (patella); | | of the calf (m.triceps surae). |
| | tibiofibular articulation (articulatio | | |
| | tibiofibularis); peroneal bone head | | |
| | ligament (ligg.capitis fibulae anterius | | |
| | et posterius); the tendons of the | | |
| | muscles of the anterior tibial muscle | | |
| | (m.tibialis anterior), the long | | |
| | extensor of the fingers (m.extensor | | |
| | digitorum longus), the long extensor | | |
| | of the thumb (m.extensor hallucis | | |
| | longus); joint capsule (capsula | | |
| | articularis), the deep bursa of the | | |
| | kneecap (bursa infrapatellaris | | |
| | profunda). | | |
| 8b | the place of application of the | 8d | the place of application of the |
| | electrode is the place of transition of | | electrode is the place of transition |
| | the distal part of the anterior tibial | | of the myogaster of the triceps |
| | muscle (m.tibialis anterior), | | muscle of the calf (m.triceps |
| | innervation (L_{IV}- S_{I}) and the long | | surae) into the Achilles tendon |
| | extensor of the fingers (m.extensor | | (tendo calcaneus (Achillis)), |
| | digitorum longus) to the shin bone | | innervation (L_{IV}-S_{I}) |
| | into the tendon, innervation (L_{IV}-S_{I}) | | |
| VIIb | the place of application of the | VIIa | the place of application of the |
| | heating element is localized by the | | heating element is localized by |
| | place of transition of the distal part of | | the place of the medial surface of |
| | the anterior tibial muscle (m.tibialis | | the ankle joint (articulatio |
| | anterior) and the long extensor of the | | tibiocruralis) and the medial |
| | fingers (m.extensor digitorum | | (deltoid) ligament (lig.mediale |
| | longus) into the tendon; as well as the | | (deltoideum)) adjacent to it; the |
| | lateral surface of the ankle joint | | tibial-navicular ligament |
| | (articulatio tibiocruralis) and the | | (lig.tibionavicularis); the anterior |
| | anterior and posterior tibial-fibular | | and posterior talus-tibial |
| | ligament (lig.tibiofibulare anterius et | | ligament (lig.tibiotalaris anterius |
| | posterius) adjacent to it; anterior and | | et posterius); the anterior and |
| | posterior astragalofibular | | posterior tibial-fibular ligament |
| | (lig.taliofiulare anterius et posterius); | | (lig.tibiofibulare anterius et |
| | the calcaneofibular ligament | | posterius); the articular surface |
| | (lig.calcaneofibulare); the articular | | of the ankle (facies articularis |
| | surface of the ankle (facies articularis | | maleoli tibiae); joint capsule |
| | maleoli fibilae); joint capsule | | (capsula articularis). |
| | (capsula articularis). | | |

TABLE 2
Schemes of interconnections between spinal segments and the organs
and body parts, for the innervation of which they are responsible

| | Region | |
|---|---|---|
| Designation | of the | |
| of thr | vertebral | Organs and parts of the body associated |
| vertebra | column | with the vertebra |
| 1 | 2 | 3 |
| C_{I} | cervical | Pituitary, inner ear, brain, sympathetic nervous |
| | | system |
| C_{II} | | Eyes, optic and auditory nerves, temporal |
| | | bones |
| C_{III} | | Cheeks, outer ear, facial nerve, teeth |
| C_{IV} | | Nose, lips, mouth, eustachean tube |
| C_{V} | | Throat ligaments |
| C_{VI} | | Muscles of the neck, forearms |
| C_{VII} | | Thyroid gland, shoulder joint, elbow joint |
| Th_{I} | Pectoral | Arms, wrists and palms, esophagus and |
| | (thoracic) | trachea |
| Th_{II} | | Heart |
| Th_{II} | | Bronchi, lungs, pleura, chest |
| Th_{VI} | | Gall bladder, bile duct |
| Th_{V} | | Liver, solar plexus |
| Th_{VI} | | Stomach |
| Th_{VII} | | Pancreas gland, dodecadactylon |
| Th_{VIII} | | Spleen, diaphragm |
| Th_{IX} | | Adrenal gland |
| Th_{X} | | Kidney |
| Th_{XI} | | Kidney |
| Th_{XII} | | Small and large intestines, inguinal rings, |
| | | fallopian tubes |
| L_{I} | lumbar | Abdominal cavity, upper thigh |
| L_{II} | | Cecal appendage |
| L_{III} | | Genital organs, bladder, knee |
| L_{IV} | | Prostate gland, calfs, feet |
| L_{V} | | Calfs, feet, toes |
| S_{I-V} | sacrum | Femoral bones, buttocks |

TABLE 3
Schemes of interconnections between agonist/antagonist muscle
pairs and the spinal segments responsible for their innervation

| | | | | The spinal segment responsible | |
|---|---|---|---|---|---|
| | | | | for innervations of agonist/ | |
| Spasmed agonist muscle | | Antagonist muscle | | antagonist muscle pairs | |
| Designation | Description | Designation | name | Designation | name |
| 1 | 2 | 3 | 4 | 5 | 6 |
| 1a, 1b | biceps muscle of the | 1c, 1d | triceps muscle of the | C_{V}- | innervation of |
| | shoulder (m. biceps | | shoulder (m. biceps | C_{VII,} | Nn. cervicales |
| | brachii) | | brachii) | C_{V}- | (cervical spine |
| | | | | C_{VII} | segment 5-8) |
| 1c, 1d | triceps muscle of the | 1a, 1b | biceps muscle of the | C_{V}- | innervation of |
| | shoulder (m. biceps | | shoulder (m. biceps | C_{VII,} | Nn. cervicales |
| | brachii) | | brachii) | C_{V}- | (cervical spine |
| | | | | C_{VII} | segment 5-8) |
| 2a, 2b | deltoid muscle (venter | 2c, 2d | teres major/minor | C_{V}- | innervation of |
| | m. deltoideus) | | muscle (mm. teres | C_{VI} | Nn. cervicales |
| | | | major et minor) | | (cervical spine |
| | | | | | segment 5-6) |
| 2c, 2d | greater pectoral | 2a, 2b | deltoid muscle | C_{V}- | innervation of |
| | muscle (mm. pectoralis | | (venter | C_{VI} | Nn. cervicales |
| | major) | | m. deltoideus) | | (cervical spine |
| | | | | | segment 5-6) |
| 3a, 3b | elbow flexor of the | 3c, 3d | ulnar extensor | C_{VII}- | innervation of |
| | wrist (m. flexor carpi | | muscle of the wrist | C_{VIII,} | Nn. cervicales |
| | ulnaris), | | (m. extensor carpi | C_{VII}- | (cervical spine |
| | superficial digital | | ulnaris), radial | Th_{I} | segment 7-8), |
| | flexor (m. digitorum | | extensor muscle of | | Nn. thoracici |
| | superfacialis), | | the wrist (m. extensor | | (thoracic spine |
| | deep digital flexor | | carpi radialis), | | segment 1) |
| | (venter m. flexor | | digital extensor | | |
| | digitorum profundus) | | (m. extensor | | |
| | | | digitorum) | | |
| 3c, 3d | ulnar extensor muscle | 3a, 3b | elbow flexor of the | C_{VII}- | innervation of |
| | of the wrist | | wrist (m. flexor carpi | C_{VIII,} | Nn. cervicales |
| | (m. extensor carpi | | ulnaris), | C_{VII}- | (cervical spine |
| | ulnaris), radial | | superficial digital flexor | Th_{I} | segment 7-8), |
| | extensor muscle of the | | (m. digitorum | | Nn. thoracici (thoracic |
| | wrist (m. extensor | | superfacialis), | | spine segment 1) |
| | carpi radialis), digital | | deep digital flexor | | |
| | extensor (m. extensor | | (venter m. flexor | | |
| | digitorum) | | digitorum profundus) | | |
| 4a, 4b | the rectus abdominis | 4c, 4d | the longissimus dorsi | Th_{I}- | innervation of |
| | muscle (m. rectus | | (m. longissimus), the | Th_{XII,} | Nn. thoracici (thoracic |
| | abdominis) | | iliocostalis muscle | L_{I} | spine segment 1-12), |
| | | | (m. iliocostalis) | | Nn. lumbales (lumbar |
| | | | | | spine segment 1) |
| 4c, 4d | the longissimus dorsi | 4a, 4b | rectus abdominis | Th_{I} - | innervation of |
| | (m. longissimus), the | | muscle (m. rectus | Th_{XII,} | Nn. thoracici (thoracic |
| | iliocostalis muscle | | abdominis) | L_{I} | spine segment 1-12), |
| | (m. iliocostalis) | | | | Nn. lumbales (lumbar |
| | | | | | spine segment 1) |
| 5a, 5b | the tailor's muscle of | 5c, 5d | the gluteus maximus | L_{II}- | innervation of |
| | the thigh (m. sartorius) | | muscle (m. gluteus | L_{III,} | Nn. lumbales (lumbar |
| | | | maximus) | L_{V}-S_{I} | spine segment 2-5), |
| | | | | | Nn. sacrales (sacral |
| | | | | | spine segment 1) |
| 5c, 5d | the tailor's muscle of | 5a, 5b | the gluteus maximus | L_{II} - | innervation of |
| | the thigh (m. sartorius) | | muscle (m. gluteus | L_{III,} | Nn. lumbales (lumbar |
| | | | maximus) | L_{V}-S_{I} | spine segment 2-5), |
| | | | | | Nn. sacrales (sacral |
| | | | | | spine segment 1) |
| 6a, 6b | the long adductor | 6c, 6d | the gluteus maximus | L_{II}- | innervation of |
| | muscle of the thigh | | muscle (m. gluteus | L_{III,} | Nn. lumbales |
| | (m. adductor longus | | medius), the wide | L_{IV}- | (lumbar spine |
| | femorale), the great | | fascia strainer | S_{I} | segment 2-4), Nn. |
| | adductor muscle | | (m. tensor fasciae | | sacrales (sacral |
| | (m. adductor magnus) | | latae) | | spinal segment 1) |
| 6c, 6d | the gluteus maximus | 6a, 6b | the long adductor | L_{II}- | innervation of |
| | muscle (m. gluteus | | muscle of the thigh | L_{III,} | Nn. lumbales |
| | medius), the wide | | (m. adductor longus | L_{IV}- | (lumbar spine |
| | fascia strainer | | femorale), the great | S_{I} | segment 2-4), Nn. |
| | (m. tensor fasciae | | adductor muscle | | sacrales (sacral |
| | latae) | | (m. adductor magnus) | | spinal segment 1) |
| 7a, 7b | the quadriceps muscle | 7c, 7d | the biceps muscle of | L_{IV}- | innervation of |
| | of the thigh | | the thigh (caput | L_{V,} S_{I} | Nn. lumbales |
| | (m. quadriceps | | longum m. biceps | | (lumbar spine |
| | femoris, m. rectus | | femori), the | | segment 4-5), Nn. |
| | femoris) | | semitendinosus | | sacrales (sacral |
| | | | muscle | | spinal segment 1) |
| | | | (m. semitendinosus), | | |
| | | | the | | |
| | | | semimembranosus | | |
| | | | muscle | | |
| | | | (m. semimembranosus), | | |
| | | | the great adductor | | |
| | | | musle (m. abductor | | |
| | | | magnus). | | |
| 7c, 7d | the quadriceps muscle | 7a, 7b | the biceps muscle of | L_{IV}- | innervation of |
| | of the thigh | | the thigh (caput | L_{V,} S_{I} | Nn. lumbales |
| | (m. quadriceps | | longum m. biceps | | (lumbar spine |
| | femoris, m. rectus | | femori), the | | segment 4-5), Nn. |
| | femoris) | | semitendinosus | | sacrales (sacral |
| | | | muscle | | spinal segment 1) |
| | | | (m. semitendinosus), | | |
| | | | the | | |
| | | | semimembranosus | | |
| | | | muscle | | |
| | | | (m. semimembranosus), | | |
| | | | the great adductor | | |
| | | | musle (m. abductor | | |
| | | | magnus). | | |
| 7a, 7b | the tibial muscle | 7c, 7d | triceps muscle of the | L_{IV}- | innervation of |
| | (m. tibialis anterior), | | calf (m. triceps surae) | S_{II} | Nn. lumbales |
| | the long extensor of | | | | (lumbar spine |
| | the fingers | | | | segment 4-5), Nn. |
| | (m. extensor digitorum | | | | sacrales (sacral |
| | longus) | | | | spinal segment 1-2) |
| 7c, 7d | triceps muscle of the | 7a, 7b | the tibial muscle | L_{IV}- | innervation of |
| | calf (m. triceps surae) | | (m. tibialis anterior), | S_{II} | Nn. lumbales |
| | | | the long extensor of | | (lumbar spine |
| | | | the fingers | | segment 4-5), Nn. |
| | | | (m. extensor | | sacrales (sacral |
| | | | digitorum longus) | | spinal segment 1-2) |

1. A method of relaxation of spasmed muscles including a stimulating electric impulse effect on relaxed antagonist muscles and a relaxation effect on spasmed agonist muscles, characterized in that an additional thermal effect by the temperature of 42° C. to 60° C. is carried out.

2. The method according to claim 1, characterized in that the relaxation effect on the spasmed agonist muscles is carried out by exposure to thermal effect of 42° C. to 60° C., while relaxed antagonist muscles are subjected to a stimulating electric impulse effect at an impulse frequency of 15 to 35 Hz, with impulse signal duration of 5 to 60 s and working signal level of 5 to 75% of the strength of the signal at which the patient feels vibration.

3. The method according to claim 1, characterized in that the effects on the agonist/antagonist muscle pairs and the segments of the spinal cord responsible for innervating the agonist/antagonist muscle pairs are carried out simultaneously.

4. The method according to claim 1, characterized in that the effects on the agonist/antagonist muscle pairs and the segments of the spinal cord responsible for innervating the agonist/antagonist muscle pairs are carried out alternately.

5. The method according to claim 1, characterized in that the effects on the agonist/antagonist muscle pairs are carried out alternately in the setting of a constant effect on the segments of the spinal cord responsible for innervating the agonist/antagonist muscle pairs.

6. The method according to claim 1, characterized in that the stimulating electric impulse effect and the relaxing effect are carried out on individual abovementioned regions of the body.

7. The method according to claim 1, characterized in that the stimulating electric impulse effect and the relaxing effect are carried out on all of the abovementioned regions of the body simultaneously.

8. The method according to claim 2, characterized in that the effects on the agonist/antagonist muscle pairs and the segments of the spinal cord responsible for innervating the agonist/antagonist muscle pairs are carried out simultaneously.

9. The method according to claim 2, characterized in that the effects on the agonist/antagonist muscle pairs and the segments of the spinal cord responsible for innervating the agonist/antagonist muscle pairs are carried out alternately.

10. The method according to claim 2, characterized in that the effects on the agonist/antagonist muscle pairs are carried out alternately in the setting of a constant effect on the segments of the spinal cord responsible for innervating the agonist/antagonist muscle pairs.

11. method according to claim 2, characterized in that the stimulating electric impulse effect and the relaxing effect are carried out on individual abovementioned regions of the body.

12. The method according to claim 2, characterized in that the stimulating electric impulse effect and the relaxing effect are carried out on all of the abovementioned regions of the body simultaneously.
